(19) United States
(12) Patent Application Publication
(10) Pub. No.: US 2019/0301987 A1
(43) Pub. Date: Oct. 3, 2019

(54) Sample Preparation for Antimicrobial Susceptibility Testing

(71) Applicant: SELUX DIAGNOSTICS, INC., Charlestown, MA (US)

(72) Inventors: Benjamin Spears, Charlestown, MA (US); Kelly Flentie, Charlestown, MA (US); Aleksandar Vacic, Charlestown, MA (US); Eric Stern, Charlestown, MA (US)

(21) Appl. No.: 16/290,663

(22) Filed: Mar. 1, 2019

Related U.S. Application Data
(60) Provisional application No. 62/637,786, filed on Mar. 2, 2018.

(51) Int. Cl. G01N 1/40

(52) U.S. Cl. CPC G01N 1/4077; G01N 33/569; G01N 2001/4083; G01N 1/4044

(57) ABSTRACT

The present disclosure provides novel methods for microbiological sample preparation from patients that yield high quality input material for antimicrobial susceptibility testing (AST) without the need for plate culture. Certain methods of this disclosure comprise multiple separation steps to purify microbial samples for use in ASTs.

### CROSS REFERENCE TO RELATED APPLICATIONS

This application claims priority to U.S. provisional application 62/637,786, filed on Mar. 2, 2018, and titled “Sample Preparation for Antimicrobial Susceptibility Testing.” The foregoing application is incorporated by reference herein in its entirety and for all purposes.

### FIELD OF THE DISCLOSURE

This disclosure relates to the preparation and testing of clinical microbiological samples.

### BACKGROUND

Antimicrobial-resistant microbial infections are associated with poor clinical outcomes including increased morbidity, mortality, and healthcare costs among infected patients. The prevalence of these organisms in the United States has steadily increased over the last 30 years. Phenotypic antimicrobial susceptibility testing (AST) of microorganisms is critical for informing physicians of appropriate therapeutic regimens. Using current methods, AST determination typically requires a minimum of eight hours, rendering it an overnight process due to shift work in many clinical microbiology laboratories. While awaiting a determination from current AST methods, patients are often administered broad-spectrum antimicrobials which often have significant detrimental effects on patient health and/or contribute to the growing antimicrobial resistance epidemic. Furthermore, this time delay obtaining accurate antimicrobial treatment information increases patient stays in hospitals, thereby increasing costs and inconvenience to the patient.

Against this backdrop, government and industry stakeholders have proposed rules to promote antimicrobial stewardship in hospitals. However, antimicrobial stewardship efforts may be complicated by the limitations of current AST methods and antimicrobial prescribing practices. The inventors have previously disclosed a method for rapid AST, that may reduce the time required for AST determinations and facilitate antimicrobial stewardship. For example, AST systems and methods utilizing fluorescent probes that bind microorganism surfaces are described in commonly owned U.S. Pat. No. 9,834,808 and PCT Publication WO 2018/119439. These systems and methods are advantageous in that they address this need in a cost-effective manner and can be compatible with existing assay hardware components.

Conventional AST methods utilize plate-based culture sample preparation to purify microbes and remove contaminants from primary samples, a process which typically requires an overnight culture. Reducing or eliminating the requirement for overnight, plate-based culture could potentially accelerate the delivery of AST results to prescribers, which in turn could improve patient outcomes. Consequently, there is a need in the field today for rapid sample preparation processes that maintain microbial pathogens in a suitable state for performing AST, while removing cells, such as blood cells, and soluble species, such as antibiotics.

### SUMMARY

The present disclosure provides systems and methods for obtaining suitable AST input material directly from patient samples and/or cultured blood samples determined to be positive for microorganism growth by established methods, such as continuous blood culture monitoring. The systems and methods described below therefore allow rapid processing of patient samples for AST and reduce the time to result for rapid AST assays. In various aspects of this disclosure, the patient sample is a human sample or processed human sample, such as urine, blood, synovial fluid, cerebrospinal fluid, sputum, bronchoalveolar lavage, nasal swab, or wound swab. Processing methods of this disclosure may utilize enzymes to degrade sample matrices, such as for sputum, or fluid to disperse sample, such as for swabs. Blood samples may also be processed by liquid culture or “blood bottles,” as known to those skilled in the art. Such samples may be “positive” blood cultures or may be from bottles incubated at 33-37° C., preferably for 4-10 hours, but not yet determined to be positive.

According to these aspects of the disclosure, methods for separating microorganisms from blood cultures are provided. The blood cultures used in certain embodiments of this disclosure may be prepared in any suitable fashion, e.g., from blood cultures that have been determined to be positive using continuous monitoring blood culture systems, such as the BACTEC® (Becton-Dickinson), the BacT/Alert® (bioMérieux), and the VersaTrek™ (Thermo Fisher)). By utilizing a lytic reagent after an initial centrifugation, an exemplary method of this disclosure is capable of suitably removing a plurality of hemolytic clots, eukaryotic cells, and eukaryotic cell fragments impact optical measurement while being easily automated. Specifically, the methods described herein overcome the need for gel-based filters, such as serum separator tubes described by Lupetti et al., or solid filters, such as membrane filter described in Machen, et al. PLoS ONE 9(2): e87870 that characterizes current art-standard methods.

In some aspects, this disclosure describes an automated method for preparing a sample suspected of comprising pathogenic microorganisms derived from a patient for antimicrobial susceptibility testing, comprising the steps of: (a) centrifuging at <1000 RCF; (b) collecting a first supernatant from step a; (c) treating the first supernatant with one or more lytic reagents; (d) centrifuging the treated first supernatant from step c at >1000 RCF; (e) removing the supernatant from step d; (f) resuspending the microorganism pellet in saline or a suitable buffer solution; (g) optionally washing the pellet one or more times by repeating steps d-e, optionally after step f pellet resuspension; and (h) quantifying the cell suspension.

Further, the lytic reagent may include but is not limited to, saponins, tritons, tweens, ammonium chloride, sorbitan esters, nonionic polyoxyethylene surfactants. The lytic reagent may comprise one or more water-soluble compounds in addition to the one or more lytic agents. The water-soluble compounds may include, but are not limited to, sodium polyanethole sulfate (SPS), polypropylene glycol (PPG), potassium carbonate, potassium bicarbonate, N-cyclohexyl-3-aminopropanesulfonic acid (CAPS), ethylenediaminetetraacetic acid (EDTA), sodium chloride. The lytic reagent may comprise saponin, SPS, and PPG. The lytic reagent may comprise ammonium chloride and potassium bicarbonate is added during lysis. The lytic reagent may comprise ammonium chloride and potassium bicarbonate and EDTA or EDTA sodium salt. The lytic reagent may comprise Brij 97 and CAPS.

Further, the pellet may not be washed. The pellet may be washed once. The wash may be performed with saline, a neutral buffer, or growth media. The pellet may be resuspended following addition of wash solution. The pellet may not be resuspended following addition of wash solution. The pellet may be washed twice. The wash may be performed with saline, a neutral buffer, or growth media. The pellet may be resuspended following addition of wash solution. The pellet may not be resuspended following addition of wash solution. The cells in the resuspended pellet may be quantified optically at one or more wavelengths between 500 nm and 700 nm. The cells may be quantified by a spectrometer, a spectrophotometer, or a nephelometer. The cells may be quantified following treatment of the suspended cells with one or more precursor compounds. The precursor compounds may include, but are not limited to, PicoGreen, acridine orange, RedSafe, 4′,6′-diamidino-2-phenylindole, ethidium bromide, SYBR green I, SYBR green II, luciferin, and resazurin. The precursor compound may be added to an aliquot of the resuspended pellet suspension and this aliquot is then measured. The first centrifugation may be 500 RCF. The second centrifugation may be 2370 RCF. The third centrifugation may be 2370 RCF. The fourth centrifugation may be 2370 RCF. One or more centrifugations may be performed at a fixed angle <90°. One or more centrifugations may be performed at a fixed angle of 30°, 45°, 60° relative to the axis of rotation.

In some aspects, the method may be performed on positive blood cultures from continuous monitoring systems. The method may be performed on cultures that turned positive ≤48 hours after introduction into the continuous monitoring blood culture system. The method may be performed on urine samples. The volume of the sample derived from the patient is less than 20 mL, 15 mL, 10 mL.

In one embodiment, this disclosure discusses an automated method for preparing a sample suspected of comprising pathogenic microorganisms derived from a patient for antimicrobial susceptibility testing comprising the steps of: (a) applying a relative centrifugal force (RCF) of <1000×g to a sample, thereby producing a particulate depleted supernatant; (b) lysing at least one patient cell or platelet in the particulate depleted supernatant, thereby producing a patient cell depleted supernatant; (c) applying an RCF of >1000×g to the patient cell depleted supernatant, thereby forming a pellet; (d) collecting and resuspending the pellet; (e) optionally washing the pellet one or more times by repeating steps c and d; and (f) assessing a density of microbes in a saline or buffered solution.

In this embodiment, the lytic reagent may include but is not limited to, saponins, tritons, tweens, ammonium chloride, sorbitan esters, nonionic polyoxyethylene surfactants. The lytic reagent may comprise one or more water-soluble compounds in addition to the one or more lytic agents. The water-soluble compounds may include, but are not limited to, sodium polyanethole sulfate (SPS), polypropylene glycol (PPG), potassium carbonate, potassium bicarbonate, N-cyclohexyl-3-aminopropanesulfonic acid (CAPS), ethylenediaminetetraacetic acid (EDTA), sodium chloride. The lytic reagent may comprise saponin, SPS, and PPG. The lytic reagent may comprise ammonium chloride and potassium bicarbonate is added during lysis. The lytic reagent may comprise ammonium chloride and potassium bicarbonate and EDTA or EDTA sodium salt. The lytic reagent may comprise Brij 97 and CAPS.

In this embodiment, the pellet may not be washed. The pellet may be washed once. The wash may be performed with saline, a neutral buffer, or growth media. The pellet may be washed twice. The wash may be performed with saline, a neutral buffer, or growth media. The pellet may be resuspended in saline, a neutral buffer, or growth media. The cells in the resuspended pellet may be assessed optically at one or more wavelengths between 500 nm and 700 nm. The cells may be assessed by a spectrometer, a spectrophotometer, or a nephelometer. The cells may be quantified following treatment of the suspended cells with one or more precursor compounds. The precursor compounds may include, but are not limited to, PicoGreen, acridine orange, RedSafe, 4′,6′-diamidino-2-phenylindole, ethidium bromide, SYBR green I, SYBR green II, luciferin, and resazurin. The precursor compound may be added to an aliquot of the resuspended pellet suspension and this aliquot is then measured. The first centrifugation may be 500 RCF. The second centrifugation may be 2370 RCF. The third centrifugation may be 2370 RCF. The fourth centrifugation may be 2370 RCF. One or more centrifugations may be performed at a fixed angle <90°. One or more centrifugations may be performed at a fixed angle of 30°, 45°, 60° relative to the axis of rotation.

In this embodiment, the method may be performed on positive blood cultures from continuous monitoring systems. The method may be performed on cultures that turned positive ≤48 hours after introduction into the continuous monitoring blood culture system. The method may be performed on urine samples. The volume of the sample derived from the patient is less than 20 mL, 15 mL, 10 mL.

In another aspect, this disclosure describes an automated method for preparing a sample suspected of comprising pathogenic microorganisms derived from a patient for antimicrobial susceptibility testing comprising the steps of: (a) applying a first relative centrifugal force to a sample, thereby producing a relatively clarified supernatant; (b) lysing at least one patient cell or platelet in the relatively clarified supernatant, thereby producing a patient cell depleted supernatant; (c) applying a second relative centrifugal force to the sample from step b, thereby forming a pellet; (d) removing the supernatant; (e) optionally washing the pellet one or more times by repeating steps c and d; (f) resuspending the pellet in a suitable saline or buffered solution; and (g) assessing a density of microbes in the saline or buffered solution.

In this aspect, the step of lysing at least one patient cell may comprise contacting the clarified supernatant with a lytic reagent selected from the group consisting of saponins, tritons, tweens, ammonium chloride, sorbitan esters, nonionic polyoxyethylene surfactants. The lytic reagent may comprise one or more water-soluble compounds in addition to the one or more lytic agents. The water-soluble compounds may include, but are not limited to, sodium polyanethole sulfate (SPS), polypropylene glycol (PPG), potassium carbonate, potassium bicarbonate, N-cyclohexyl-3-aminopropanesulfonic acid (CAPS), ethylenediaminetetraacetic acid (EDTA), sodium chloride. The lytic reagent may comprise saponin, SPS, and PPG. The lytic reagent may comprise ammonium chloride and potassium bicarbonate is added during lysis. The lytic reagent may comprise ammonium chloride and potassium bicarbonate and EDTA or EDTA sodium salt. The lytic reagent may comprise Brij 97 and CAPS.

In this aspect, the pellet may not be washed. The pellet may be washed once. The wash may be performed with saline, a neutral buffer, or growth media. The pellet may be washed twice. The wash may be performed with saline, a neutral buffer, or growth media. The pellet may be resuspended in saline, a neutral buffer, or growth media. The cells in the resuspended pellet may be assessed optically at one or more wavelengths between 500 nm and 700 nm. The cells may be assessed by a spectrometer, a spectrophotometer, or a nephelometer. The cells may be quantified following treatment of the suspended cells with one or more precursor compounds. The precursor compounds may include, but are not limited to, PicoGreen, acridine orange, RedSafe, 4′,6′-diamidino-2-phenylindole, ethidium bromide, SYBR green I, SYBR green II, luciferin, and resazurin. The precursor compound may be added to an aliquot of the resuspended pellet suspension and this aliquot is then measured. The first RCF applied may be lower than the second RCF applied. The first RCF may be 500 RCF. The second RCF may be 2370 RCF. The third RCF may be 2370 RCF. The fourth RCF may be 2370 RCF. One or more RCFs may be performed at a fixed angle <90°.

In this embodiment, the method may be performed on positive blood cultures from continuous monitoring systems. The method may be performed on cultures that turned positive ≤48 hours after introduction into the continuous monitoring blood culture system. The volume of the sample derived from the patient may be less than 10 mL.

In another embodiment, this disclosure describes a method for preparing a sample suspected of comprising pathogenic microorganisms derived from a patient for antimicrobial susceptibility testing comprising the steps of: (a) centrifuging at <1000×g; (b) collecting the supernatant from step a, followed by centrifuging at >1000×g; (c) cleaning the pellet resulting from step b; (d) resuspending the pellet; and (e) inoculating microbial cells from the resuspended pellet into each of a plurality of wells of an AST cassette. The method may further comprise incubating the resuspended cells under conditions promoting microorganism growth. The pellet may be cleaned by inverted centrifugation. The pellet may be cleaned by resuspension followed by an additional centrifugation step, after which the supernatant is removed. The pellet may be resuspended in a growth media. The sample may be aliquoted into two or more independent reservoirs after pellet resuspension. The measurement of cell concentration may be performed optically by absorbance or nephelometry. The sample may be urine, blood, synovial fluid, cerebrospinal fluid, sputum, bronchoalveolar lavage, nasal swab, or wound swab. The sample may be blood that has been cultured in liquid growth media. The conditions promoting microorganism growth may comprise a temperature of 33-37° C. The conditions promoting microorganism growth comprise agitation.

Also in this disclosure is an embodiment of a method for preparing a sample suspected of comprising pathogenic microorganisms derived from a patient for antimicrobial susceptibility testing comprising the steps of: (a) placing of a sample within a centrifuge tube outfitted with a separation membrane; (b) centrifuging at <1000×g; (c) washing of the membrane to resuspend the deposited bacteria; and (d) measuring the resuspended cell concentration. The method further comprises incubating the resuspended cells under conditions promoting microorganism growth. The sample may be aliquoted into two or more independent reservoirs after bacteria resuspension. The measurement of cell concentration may be performed optically by absorbance or nephelometry. The sample may be urine, blood, synovial fluid, cerebrospinal fluid, sputum, bronchoalveolar lavage, nasal swab, or wound swab. The sample may be blood that has been cultured in liquid growth media. The conditions promoting microorganism growth may comprise a temperature of 33-37° C. The conditions promoting microorganism growth may comprise agitation.

This disclosure further discusses a method for preparing a sample suspected of comprising pathogenic microorganisms derived from a patient for antimicrobial susceptibility testing comprising the steps of: (a) applying a first relative centrifugal force to a sample, thereby forming a first pellet and a first supernatant; (b) applying a second relative centrifugal force to the first supernatant, thereby a second pellet and a second supernatant; (c) cleaning the second pellet resulting from step b; (d) resuspending the second pellet in a buffered solution; and (e) measuring a concentration of microorganisms resuspended in the buffered solution. This method further comprises incubating the resuspended microorganisms under conditions promoting microorganism growth. The first relative centrifugal force may be lower than the second relative centrifugal force. The pellet may be cleaned by inverted centrifugation. The pellet may be cleaned by resuspension followed by an additional centrifugation step, after which the supernatant may be removed. The pellet may be resuspended in a growth media. The sample may be aliquoted into two or more independent reservoirs after pellet resuspension. The measurement of cell concentration may be performed optically by absorbance or nephelometry. The sample may be urine, blood, synovial fluid, cerebrospinal fluid, sputum, bronchoalveolar lavage, nasal swab, or wound swab. The sample may be blood that has been cultured in liquid growth media. The conditions promoting microorganism growth may comprise a temperature of 33-37° C. The conditions promoting microorganism growth may comprise agitation.

In yet another embodiment, this disclosure discusses an in vitro microbial separation system for preparing a sample comprising pathogenic microorganisms derived from a patient for antimicrobial susceptibility testing comprising: a fluid handling module configured to enable solution addition to and removal from the sample; a centrifuge having a longitudinal axis extending through a fixed rotor of the centrifuge, a normal axis that is normal to the longitudinal axis, and an angle of the fixed rotor; a vortexer configured to agitate the sample; and a cell quantification apparatus configured to measure a metric of the sample. The angle may be about 30°, 45°, 60° from the normal axis.

This disclosure also discusses method for preparing a sample comprising pathogenic microorganisms derived from a patient for antimicrobial susceptibility testing comprising: transferring the sample via a fluid handling module into a sample tube; disposing the sample tube into a centrifuge having a fixed rotor at an angle with respect to a normal axis that is normal to a longitudinal axis of the centrifuge; activating the centrifuge for a period of time; removing a portion of a fluid of the sample from the sample tube; adding a portion of lytic reagent to the sample tube; subjecting the sample tube to a vortexer configured to agitate the sample; adding a portion of saline or buffered solution to the sample tube; transferring substantially all of the fluid in the sample tube to a cuvette; measuring a metric of the sample using an optical cell quantification apparatus at one or more wavelengths between 500 nm and 700 nm; diluting the suspension to a pre-defined level; verifying a metric of the sample using the cell quantification apparatus; and transferring the fluid to an output tube.

### BRIEF DESCRIPTION OF THE DRAWINGS

FIG. 1 depicts a protocol for the preparation of blood.

FIG. 2 depicts a protocol for the preparation of urine.

FIG. 3 depicts a protocol for an alternate preparation of urine.

FIG. 4 depicts a protocol for an alternate preparation of blood.

FIG. 5 depicts a protocol for the automated process.

FIG. 6 depicts the subcomponents of an apparatus for preparing a sample comprising pathogenic microorganisms derived from a patient for AST including a fluid handling module configured to contain the sample.

FIG. 7 panels a-f depict representative data for E. coli sample 1 without lytic reagent.

FIG. 8 panels a-f depict representative data for E. coli sample 2 without lytic reagent.

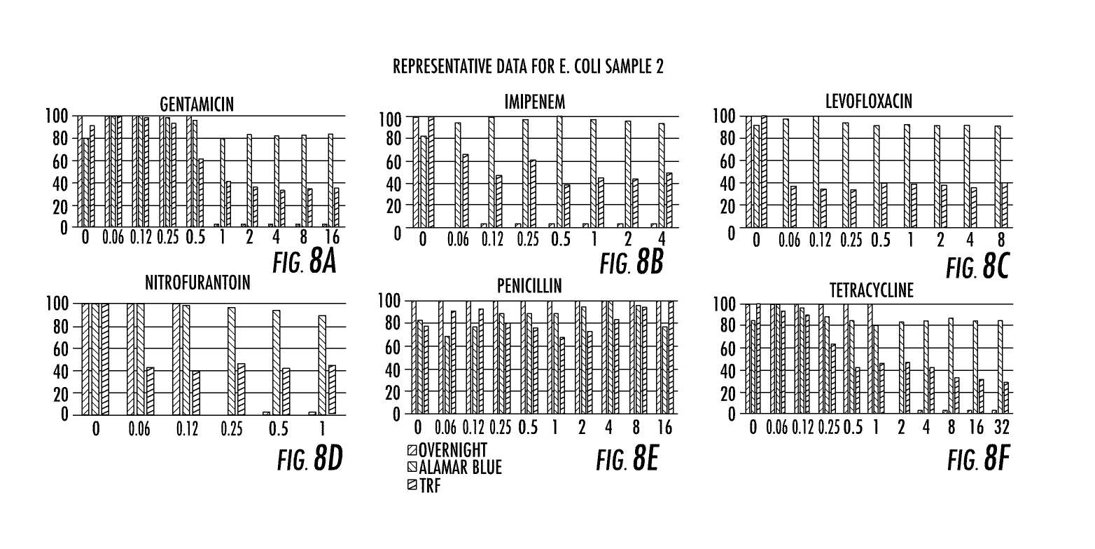

FIG. 9 panels a-f depict representative data for K. pneumoniae without lytic reagent.

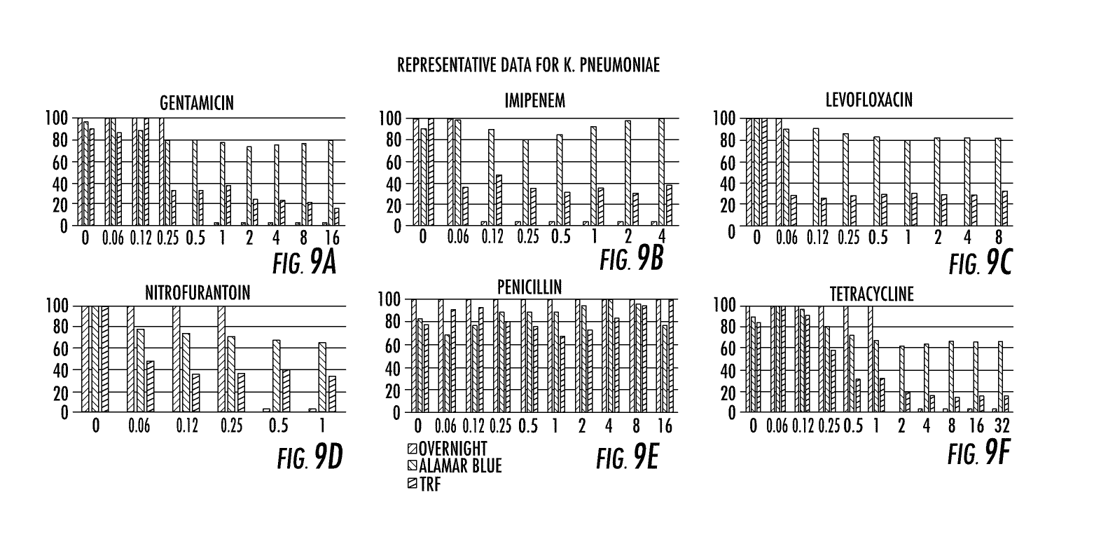

FIG. 10 panels a-d depict the results of a bacterial centrifugation experiment, showing the relationship between RCF, centrifugation time and cell concentration.

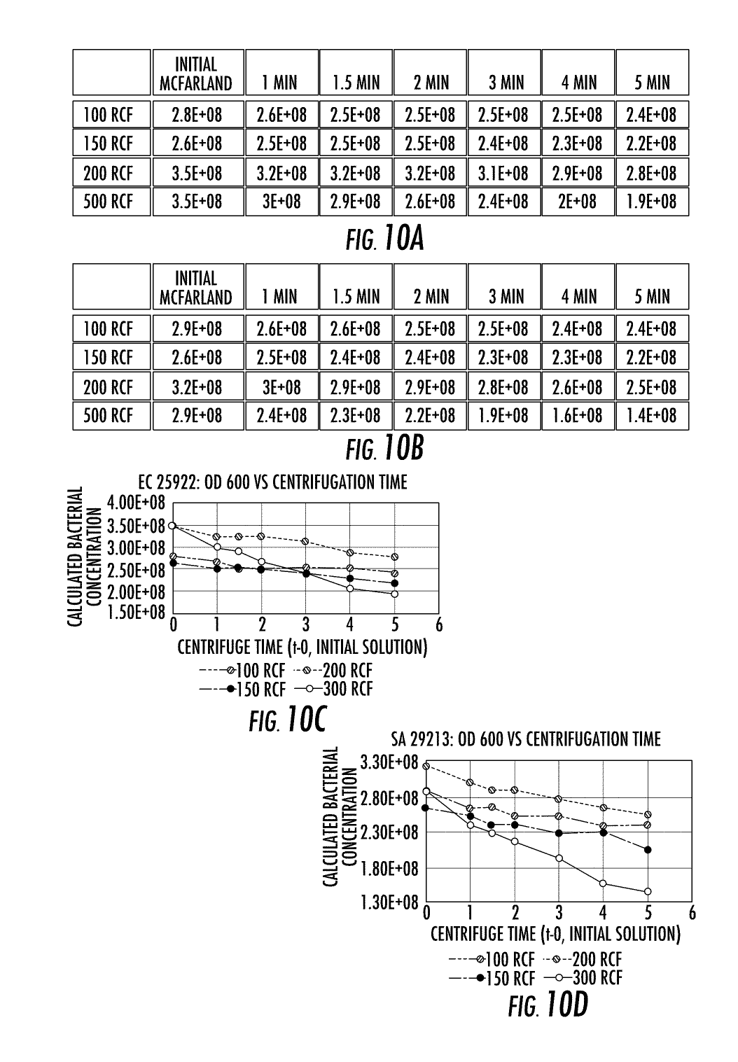

FIG. 11 depicts the formation of microbial pellets observed at varying angles relative to the plane of the rotor, from left to right 90°, 75°, 60°, 45°, 30° and 15°.

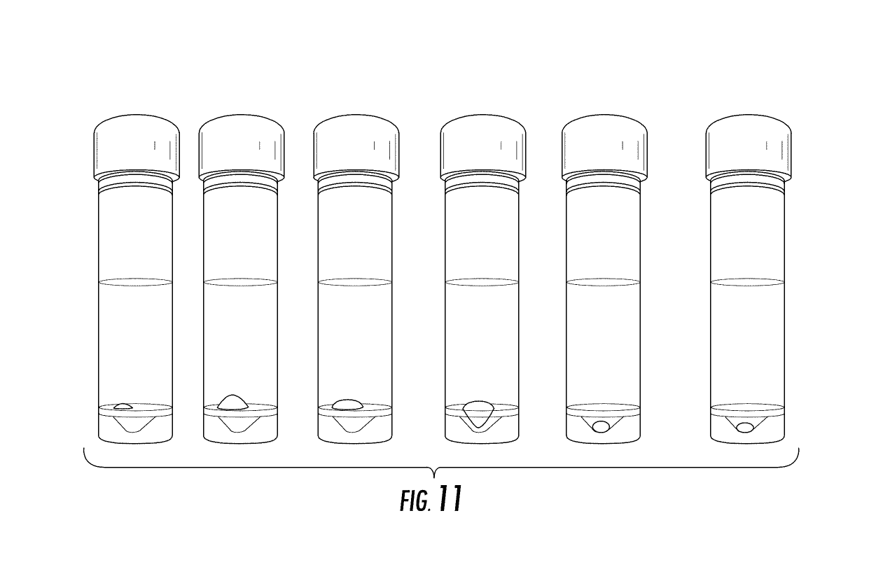

FIG. 12 panels a-f depict representative data for E. coli sample 1 with lytic reagent.

FIG. 13 panels a-f depict representative data for S. aureus with lytic reagent.

FIG. 14 panels a-f depict representative data for S. aureus without lytic reagent.

FIG. 15 panels a-f depict representative data for E. faecium without lytic reagent.

FIG. 16a-d depicts various methods of separation.

FIG. 17 depicts the representative data for the methods of FIG. 16.

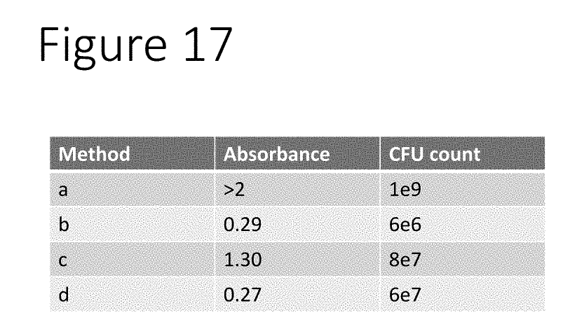

### DETAILED DESCRIPTION

### Preparation of Patient Microbiological Samples

This disclosure provides new systems and methods for separating microorganisms from primary and liquid-cultured patient samples such as blood cultures, urine samples, sputum samples, etc. Some separation methods according to this disclosure employ an initial slow centrifugation step followed by application of a lytic reagent and are capable of suitably removing a plurality of hemolytic clots, eukaryotic cells and eukaryotic cell fragments, and other constituents that may impact optical measurements of cell concentration as well as AST results. The methods further produce a “clean” sample comprising a plurality of viable microorganisms at sufficient numbers and concentration to perform AST from ≤20 mL, preferably ≤10 mL, of a positive blood culture or urine sample. At the same time, the methods of this disclosure are simple enough to be easily automated. Embodiments of this disclosure may also obviate the need for filtration of samples (e.g., using serum separator tubes described by Lupetti et al., or solid filters, such as membrane filter described in Machen, et al. PLoS ONE 9(2): e87870), potentially easing automation and lowering consumable costs.

In some embodiments, methods of this disclosure are applied to blood cultures that have been determined to be positive using continuous monitoring blood culture systems, such as the BACTEC® (Becton-Dickinson), the BacT/Alert® (bioMérieux), and the VersaTrek™ (Thermo Fisher).

It may be advantageous, particularly when preparing a blood culture or urine sample for AST, to separate microbial cells from eukaryotic cells and eukaryotic cell fragments, and large non-cellular debris derived from the patient, as well as from soluble immune peptides and antibiotics that may be present (Lupetti, et al. Clin. Microbiol. Infect. 2010; 16: 986-991). Cells and cell fragments may compromise AST result integrity and may also complicate or inhibit accurate microorganism enumeration prior to the onset of AST. Accurate microorganism enumeration through optical density and/or nephelometry may be inhibited by optically-active components, such as hemolytic clots and platelets (Lupetti, et al.).

The literature teaches that lytic reagents accomplish the same thing as a slow centrifugation or filter, namely removing eukaryotic cells and debris (e.g. platelets, hemolytic clots, etc.) from samples. These studies suggest that the use of both lytic reagents and centrifugation or filtration are redundant, and the predominant view in the field today is that a lytic reagent alone, when applied to a positive blood culture, is sufficient to remove eukaryotic cells and debris.

Some embodiments of this disclosure rest on the inventors' discovery that, by following a slow centrifugation with a chemical lysis on positive blood cultures, a final microorganism-comprising sample is produced which is sufficiently free of eukaryotic cells and debris to enable accurate microbial enumeration using optical measurements, while yielding a high-quality input material for downstream AST assays. In particular, this approach may require ≤10 mL of positive blood culture or urine to produce a sample comprising a sufficient number and suitable quantity and concentration of a plurality of viable microorganisms to enable AST. More particularly, certain embodiments of this disclosure relate to automatable methods for producing concentrated microorganism-comprising samples with a McFarland reading of approximately 0.2-0.65 from positive blood cultures without the need for an intervening plate-based culture. Those skilled in the art will appreciate that, though the CLSI broth microdilution reference method utilizes a McFarland reading of 0.5, this is diluted 200-fold for AST testing thus lower inoculum concentrations may be utilized for AST provided suitable volumes are present.

More generally, some embodiments of this disclosure relate to systems and methods for the preparation of clinical microbial inoculates directly from patient samples without the need for additional purification by intervening plate-based culture.

An exemplary method of the present disclosure comprises four primary steps. A first separation step (such as a slow centrifugation) is utilized to separate (e.g., pellet) hemolytic clots and other optically-interfering bodies from the remaining sample material (e.g., the supernatant). The clarified fluid material resulting from the first separation is treated with a lytic reagent, which lyses remaining eukaryotic cells and/or eukaryotic cell fragments, platelets, etc. while leaving intact the microorganisms that will be interrogated in a downstream assay (e.g., an AST assay). The term “lytic reagent” as used here encompasses the use of detergents, such as saponins, that may create irreversible pores in cells but leave the cell shape intact, termed “saponin ghosts” in the literature. Then, a second separation step (such as a fast centrifugation) is utilized to separate (e.g., pellet) the microorganism from the treated supernatant, followed by removal of the supernatant by decanting, aspiration, etc. Optionally, one or more wash steps may be performed to clean the pellet and remove undesired soluble species. Finally, the microorganisms of interest are resuspended in saline, buffer, or growth media and the cell concentration is determined, e.g., by optical measurement.

The first separation is designed to remove contaminating species that will interfere with optical measurements of microbial cell density. In practice, this can include separation (e.g., pelleting, collection, etc.) of eukaryotic cells and cell fragments and insoluble materials (e.g. clots, eukaryotic cell aggregates and debris) from the microbes that will be interrogated in a downstream process such as AST. In various embodiments, these separations are achieved by, for example, centrifugation.

When centrifugation is used for the first separation (referred to as a “first centrifugation”), the speed of the first centrifugation (i.e., the relative centrifugal force or RCF) is preferentially set to be sufficiently high to collect eukaryotic cells and debris while sufficiently low to enable a plurality of microorganisms and bacterial clusters, such as those formed by Staphylococcus aureus, to remain in suspension. In some embodiments, the speed is preferably under 1000 RCF, e.g., 900, 800, 700, 600, 500, 400, 300, 200 or 100 RCF.

A lytic reagent may optionally be used following the first centrifugation to further reduce the presence of non-microbial contaminating species, e.g., by lysis of eukaryotic cells and eukaryotic cell fragments (e.g., platelets) while leaving the microbes that will be interrogated by a downstream process substantially intact. The term lytic reagent can encompass one or more lytic agents including, but not limited to, saponins, ammonium chloride, triton X, sorbitan esters (e.g., Span), tweens, and nonionic polyoxyethylene 10 oleoyl ether surfactants (e.g., Brij (commercialized by Sigma-Aldrich)). The lytic reagent may further comprise one or more additional water-soluble compounds including, but not limited to, sodium polyanethole sulfonate (SPS), polypropylene glycol (PPG), potassium bicarbonate, sodium bicarbonate, potassium carbonate, N-cyclohexyl-3-aminopropanesulfonic acid (CAPS), ethylenediaminetetraacetic acid, and other salts. Treatment time with the lytic reagent is preferably <20 mins, most preferably <5 mins. In an exemplary embodiment, the lytic agent comprises saponin, SPS, and PPG.

The second separation is designed to separate microbial species from remaining contaminant species as well as any byproducts of the optional lysis step described above. In some embodiments of the disclosure, the second separation produces a microbe-enriched input material for downstream assays, such as AST, which is substantially free of contaminating species that may interfere with optical measurements of microbe density or which may affect microbial growth. When centrifugation is used for the second separation, the speed of the second centrifugation is set sufficiently high to pellet microorganisms and sufficiently low to maintain maximal microorganism viability. In some embodiments, the speed produces an RCF greater than 1000×g, e.g., 1100, 1200, 1250, 1500, 1750, 2000, 2250, 2370 or 2500×g.

The pellets are then optionally washed one or more times. Washing decreases the concentrations of soluble species from the blood culture as well as the lytic reagent, since removal of the supernatant under practical conditions cannot be complete. The pellet may be resuspended in wash solution prior to separation. The wash solution may be saline, buffer, or growth media. In an exemplary embodiment, two washes are performed, each with 1 mL saline and without pellet resuspension. In a second exemplary embodiment, one wash is performed with 1 mL saline and with pellet resuspension in the wash buffer.

When centrifugation is used, the speeds of the optional third and further centrifugations are optionally the same as that of the second centrifugation.

After the final pellet is resuspended in saline, buffer, or growth media, cell quantification is performed. This may be beneficial to ensure subsequent AST is performed with an appropriate cell concentration. Cells are preferably quantified optically by optical density or nephelometry measurements. Achieving sufficient cells for such measurements may require starting sample volumes of 2-20 mL, preferably 4-10 mL, from most positive blood cultures from BACTEC or BacT/Alert bottles. The desired final cell concentration may be determined to be approximately 0.5 McFarland, as known to those skilled in the art.

In an exemplary embodiment, the pellet resulting from a third or fourth separation is resuspended in 1 mL saline. An optical density (OD) measurement is then performed using a spectrophotometer at one or more wavelengths between 500-700 nm. If a measurement corresponding to a McFarland >0.65 is obtained, additional saline may be added to dilute the sample. The volume of additional saline may be determined by the OD measurement such that larger volumes are added for greater OD values. A subsequent OD measurement may then be taken. This measurement followed by dilution process may be performed one or more times.

In an exemplary embodiment, an OD measurement that corresponds to a McFarland <0.5 may be diluted to achieve a McFarland concentration of 0.2 or lower. This information may then be sent electronically to the AST platform to ensure proper dilutions are achieved during panel inoculation. Alternatively, an OD measurement that corresponds to a McFarland <0.5 may be flagged to the user that insufficient microorganisms are present.

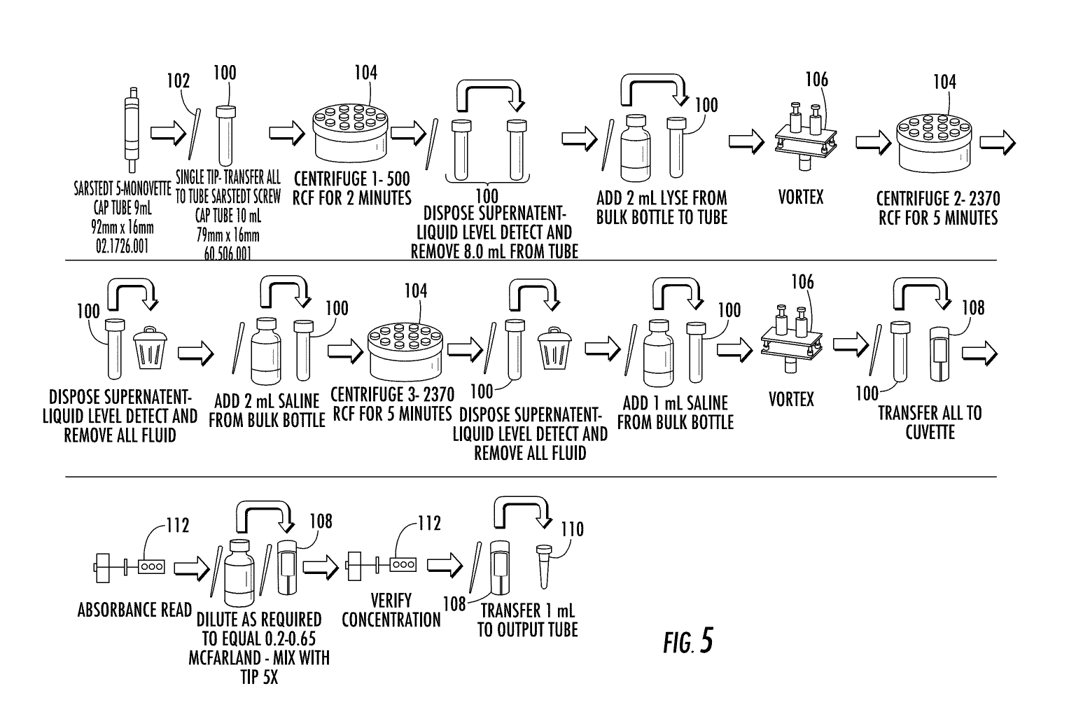

FIG. 5 shows the utility of an exemplary method to produce McFarland suspensions from blood cultures.

Alternatively, an aliquot of the resuspended pellet may be made followed by treatment with a precursor reagent that enables cell quantification. Such reagents include, but are not limited to, PicoGreen, acridine orange, RedSafe, 4′,6′-diamidino-2-phenylindole, ethidium bromide, SYBR green I, SYBR green II, luciferin, and resazurin. In this embodiment, the addition of the lytic reagent may not be necessary.

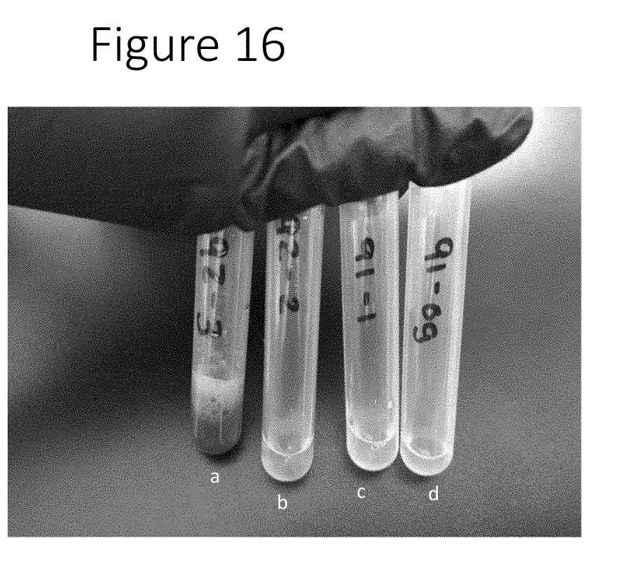

The importance of the methods are highlighted by the fact that samples obtained with the same steps but in a different order are not sufficiently clear of eukaryotic cells and debris: sample treatment with the lytic reagent, followed by the slow spin, followed by supernatant transfer to a fresh tube, followed by a fast spin to pellet microorganisms, followed by optional washes and cell quantification. Result seen in FIG. 16C.

The importance of the methods are further highlighted by the fact that samples obtained with similar steps but in a different order are not sufficiently clear of eukaryotic cells and debris: sample treatment with the lytic reagent, followed by a fast spin to pellet microorganisms, followed by supernatant removal and pellet resuspension in wash solution, followed by a slow spin, followed by supernatant transfer to a fresh tube, followed by a fast spin to pellet microorganisms, followed by optional washes and cell quantification. Result seen in FIG. 16B.

The importance the methods are further highlighted by the fact that samples obtained with similar steps but in a different order are not sufficiently clear of eukaryotic cells and debris: sample spun at top speed to sediment everything present, treatment with different lytic reagents, followed by a fast spin to pellet microorganisms, followed by supernatant removal and pellet resuspension in wash solution, followed by a fast spin to pellet microorganisms, followed by optional washes and cell quantification. Result seen in FIG. 16A.

In alternate embodiments, the sample is first centrifuged at a speed sufficient to sediment eukaryotic cells but insufficient to sediment microbes, <1000 RCF. The supernatant is collected and centrifuged at a speed sufficient to sediment microbes, >1000 RCF. The supernatant is decanted, the tube is inverted, and centrifuged at low speed, <1000 RCF, to dry the pellet. The pellet is then resuspended into a microorganism growth media, such as Mueller-Hinton Broth (MHB), forming the “processed sample.” Optical density, fluorescence, and/or nephelometry measurements are then performed to determine cell concentration. If insufficient for a reading, additional incubations may be performed. After sufficient cell concentration is achieved, such as a 0.5 McFarland concentration, as known to those skilled in the art, the sample may be aliquoted into wells of a cartridge for performing AST.

In an alternative embodiment, the sample is first centrifuged at a speed and time sufficient to sediment all cells with smaller cells moving past spaces in bigger cells to the bottom of the pellet, >1000 RCF for >5 mins. The supernatant is decanted and the large-cell pellet is aspirated. The remaining pellet is resuspended and centrifuged at a speed sufficient to sediment eukaryotic cells but insufficient to sediment microbes, <1000 RCF. The supernatant is collected and centrifuged at a speed sufficient to sediment microbes, >1000 RCF. The pellet is then resuspended into a microorganism growth media, such as Mueller-Hinton Broth (MHB), forming the processed sample. Optical density, fluorescence, and/or nephelometry measurements are then performed to determine cell concentration. If insufficient for a reading, additional incubations may be performed. After sufficient cell concentration is achieved, such as a 0.5 McFarland concentration, as known to those skilled in the art, the sample may be aliquoted into wells of a cartridge for performing AST.

In an alternative embodiment, the sample is first placed with into a tube outfitted with a separation membrane and centrifuged at a speed sufficient to pull the sample through the membrane, depositing the bacteria onto the surface of the membrane. The membrane is then washed with a microorganism growth media, such as MHB, to resuspend the deposited bacteria. Optical density, fluorescence, and/or nephelometry measurements are then performed to determine cell concentration. If insufficient for a reading, additional incubations may be performed. After sufficient cell concentration is achieved, such as a 0.5 McFarland concentration, as known to those skilled in the art, the sample may be aliquoted into wells of a cartridge for performing AST.

In various embodiments described herein or otherwise, methods for preparing a sample comprising pathogenic microorganisms derived from a patient for AST may performed by using an apparatus or system described herein. Referring to FIG. 5, an automated or semi-automated system may be used to perform the steps of the methods. This system may comprise a fluid handling module 102 that is configured to transport a sample into a sample tube 100 configured to contain the sample. The fluid handling component may be an automated liquid handler or the like. The fluid handling component may be configured such that fluids, containers, tubes, and/or samples are moved throughout the systems automatically with little or no operator handling required. This automation may increase throughput while reducing labor time and errors. The sample tube 100 containing the sample is transferred to a centrifuge 104. The centrifuge 104 has a fixed rotor configured to hold sample tubes at an angle other than 90° relative to a plane of the rotor of, which plane comprises a longitudinal axis due to the symmetry of centrifuge rotors. The angle may be any angle, e.g., about 30°, about 45°, about 60°, etc. with respect to the plane of the rotor. As described below the angles utilized in this disclosure result in the formation of a well-defined pellet that is positioned away from the bottom of the tube, permitting automated aspiration of fluid following centrifugation without undue risk of loss of the microbial material of interest. The centrifuge 104 is activated for a desired period of time. After centrifugal separation, a portion of a fluid of the sample is removed from the sample tube 100. A portion of lytic reagent is then added to the sample tube 100. The sample tube 100 is then subjected to a vortexer 106 to agitate and mix the contents of the sample tube 100. After agitation and mixing, the sample tube 100 is added to the centrifuge 104 and is subjected to centrifugal separation. After separation, at least a portion of the fluid contained in the sample tube 100 is removed. Then, a portion of saline is added to the sample tube 100. The sample tube 100 is then placed into the centrifuge 104 and is subjected to centrifugal separation. After separation, at least a portion of the fluid contained in the sample tube 100 is removed. A portion of saline is then added to the sample tube 100. The sample tube is then subjected to the vortexer 106 to agitate and mix the contents of the sample tube 100. After agitation and mixing, substantially all of the fluid in the sample tube 100 is transferred to a cuvette 108. A cell quantification apparatus 112, e.g., a spectrometer, is then used to read an absorbance metric of the fluid within the cuvette 108. The fluid within the cuvette is then diluted to a desired dilution level. The fluid within the cuvette 108 is then measured by the cell quantification apparatus 112 to verify a concentration level of the fluid within the cuvette 108. The fluid is then transferred from the cuvette 108 into an output tube 110.

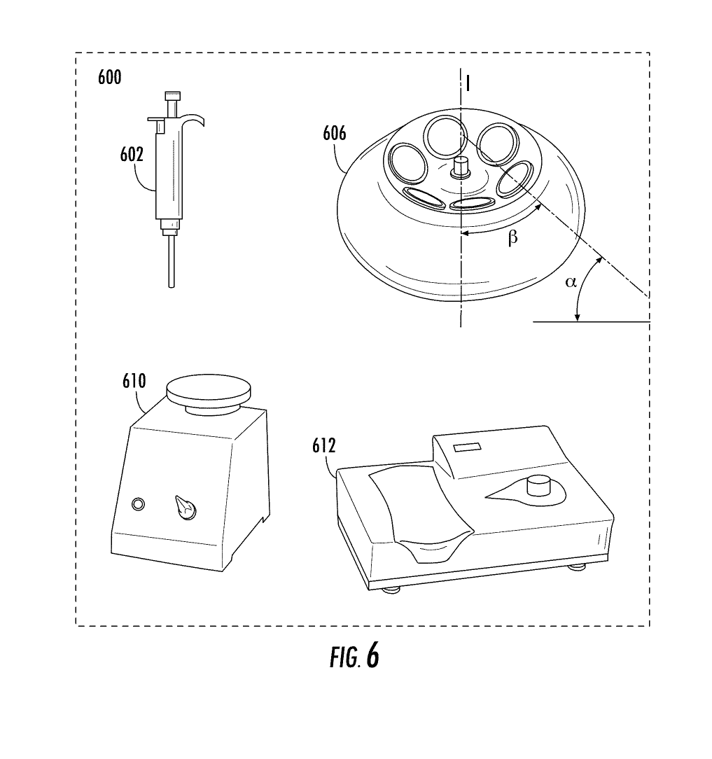

In various embodiments described herein or otherwise, methods for preparing a sample comprising pathogenic microorganisms derived from a patient for AST may performed by using an apparatus or system described herein. FIG. 6 illustrates an apparatus 600 for preparing a sample comprising pathogenic microorganisms derived from a patient for AST including a fluid handling module 602 configured to transport a sample, e.g., to a sample tube. The fluid handling module 602 may be placed into a centrifuge 606. The centrifuge 606 has a fixed rotor with a longitudinal axis l extending therethrough and a normal axis n that is normal to the longitudinal axis. The centrifuge 606 receives samples at an angle α with respect to the normal axis n and/or at another angle β with respect to the longitudinal axis l. The angles α, β may be any angle, e.g., about 30°, about 60°, or the like. A vortexer 610 may be actuated to impart an orbital motion to shake a sample. A cell quantification apparatus 612, e.g., a spectrometer may be used to read an absorbance metric of the sample.

Much of this disclosure has discussed a method which includes a lysis reagent step. However, the inventors have also found that if this lysis reagent step is omitted, the resulting sample may be suitable for instances such as direct AST testing, in which there is no need for optical quantification of microbial concentration. In an exemplary embodiment of such a method, centrifugation may be used to separate certain contaminating species (e.g., a slow centrifugation step may be used to reduce clotted material or other large extracellular debris, and/or a fast centrifugation step may be used to pellet microbes for resuspension). In this case, it may be important to determine bacterial growth for AST with one or more viability indicators, such as resazurin. Exemplary methods are disclosed in PCT Publication WO 2018/119439 paragraphs [0008]-[0134], which is incorporated by reference herein.

The exemplary embodiments described above have generally utilize centrifugation for separations. However those of skill in the art will appreciate that other separation approaches may be compatible with certain embodiments of this disclosure. For example, some methods of this disclosure may utilize filtration to separate microbial species to be interrogated by AST from contaminating species in the sample. In some embodiments, a first filtration step may have a high molecular weight cutoff. In some cases, a second filtration step may have a lower molecular weight cutoff than the first.

The foregoing disclosure has focused on embodiments of systems and methods for preparing microbial samples for, e.g., AST from patient samples. Those of skill in the art will appreciate that the methods described above, while superficially similar to those currently in use, differ in that they overcome, for the first time, a technical challenge that contributes significant delays to current AST sample preparation protocols. Namely, the methods of this disclosure yield high-quality, quantifiable input material for rapid AST without the need for an intervening plate culture step. Importantly, while also allowing for the separation of microorganisms and bacterial clusters from similarly sized eukaryotic cells and debris. In the first separation, the speed is high enough to collect eukaryotic cells and debris while low enough to enable microorganisms and bacterial clusters, such as those formed by Staphylococcus aureus, to remain in suspension. Bacteria such as Staphylococcus aureus tend to aggregate and as such may be similar in size to unwanted eukaryotic cells or fragments (e.g., platelets). Therefore, a lytic reagent may be introduced which will disrupt the lipid membranes of the eukaryotic cells remaining after the initial slow centrifugation, allowing for their removal with the second, faster separation step, producing a microbe-enriched input material for downstream assays, such as AST, which is substantially free of contaminating species that may interfere with optical measurements of microbe density or which may affect microbial growth.

As discussed above, the methods are distinct both in the steps themselves as well as the order in which they are performed. This highlights the inventive nature of these methods, as it is only through this combination of steps that the need for an intervening plate culture step may be removed, eliminating the need to spend time and resources culturing the microorganisms present in the sample, a current hindrance for similar systems.

### Microorganisms

An infection can include any infectious agent of a microbial origin, e.g., a bacterium, a fungal cell, an archaeon, and a protozoan. In some examples, the infectious agent is a bacterium, e.g., a gram-positive bacterium, a gram-negative bacterium, and an atypical bacterium. An antimicrobial resistant microorganism can be a microorganism that is resistant to an antimicrobial, i.e., anti-bacterial drugs, antifungal drugs, anti-archaea medications, and anti-protozoan drugs.

The microorganisms (e.g., a liquid suspension of microorganisms) may include one strain of microorganism. The microorganisms may include one species of microorganism. The microorganisms may include more than one strain of microorganism. The microorganisms may include one order of microorganism. The microorganisms may include one class of microorganism. The microorganisms may include one family of microorganism. The microorganisms may include one kingdom of microorganism.

The microorganisms (e.g., a liquid suspension of microorganisms) may include more than one strain of microorganism. The microorganisms may include more than one species of microorganism. The microorganisms may include more than one genus of microorganism. The microorganisms may include more than one order of microorganism. The microorganisms may include more than one class of microorganism. The microorganisms may include more than one family of microorganism. The microorganisms may include more than one kingdom of microorganism.

The microorganism may be a bacterium. Examples of bacterium include, but are not limited to, Acetobacter aurantius, Acinetobacter bitumen, Acinetobacter spp., Actinomyces israelii, Actinomyces spp., Aerococcus spp., Agrobacterium radiobacter, Agrobacterium tumefaciens, Anaplasma, Anaplasma phagocytophilum, Azorhizobium caulinodans, Azotobacter vinelandii, Bacillus, Bacillus anthracis, Bacillus brevis, Bacillus cereus, Bacillus fusiformis, Bacillus licheniformis, Bacillus megaterium, Bacillus mycoides, Bacillus spp., Bacillus stearothermophilus, Bacillus subtilis, Bacillus Thuringiensis, Bacteroides, Bacteroides fragilis, Bacteroides gingivalis, Bacteroides melaninogenicus (also known as Prevotella melaninogenica), Bartonella, Bartonella henselae, Bartonella quintana, Bartonella spp., Bordetella, Bordetella bronchiseptica, Bordetella pertussis, Bordetella spp., Borrelia burgdorferi, Brucella, Brucella abortus, Brucella melitensis, Brucella spp., Brucella suis, Burkholderia, Burkholderia cepacia, Burkholderia mallei, Burkholderia pseudomallei, Calymmatobacterium granulomatis, Campylobacter, Campylobacter coli, Campylobacter fetus, Campylobacter jejuni, Campylobacter pylori, Campylobacter spp., Chlamydia, Chlamydia spp., Chlamydia trachomatis, Chlamydophila, Chlamydophila pneumoniae (previously called Chlamydia pneumoniae), Chlamydophila psittaci (previously called Chlamydia psittaci), Chlamydophila spp., Clostridium, Clostridium botulinum, Clostridium difficile, Clostridium perfringens (previously called Clostridium welchii), Clostridium spp., Clostridium tetani, Corynebacterium, Corynebacterium diphtheriae, Corynebacterium fusiforme, Corynebacterium spp., Coxiella burnetii, Ehrlichia chaffeensis, Ehrlichia spp., Enterobacter cloacae, Enterobacter spp., Enterococcus, Enterococcus avium, Enterococcus durans, Enterococcus faecalis, Enterococcus faecium, Enterococcus galllinarum, Enterococcus maloratus, Enterococcus spp., Escherichia coli, Francisella spp., Francisella tularensis, Fusobacterium nucleatum, Gardenerella spp., Gardnerella vaginalis, Haemophilius spp., Haemophilus, Haemophilus ducreyi, Haemophilus influenzae, Haemophilus parainfluenzae, Haemophilus pertussis, Haemophilus vaginalis, Helicobacter pylori, Helicobacter spp., Klebsiella pneumoniae, Klebsiella spp., Lactobacillus, Lactobacillus acidophilus, Lactobacillus bulgaricus, Lactobacillus casei, Lactobacillus spp., Lactococcus lactis, Legionella pneumophila, Legionella spp., Leptospira spp., Listeria monocytogenes, Listeria spp., Methanobacterium extroquens, Microbacterium multiforme, Micrococcus luteus, Moraxella catarrhalis, Mycobacterium, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium spp., Mycobacterium tuberculosis, Mycoplasma, Mycoplasma fermentans, Mycoplasma genitalium, Mycoplasma hominis, Mycoplasma penetrans, Mycoplasma pneumoniae, Mycoplasma spp., Neisseria, Neisseria gonorrhoeae, Neisseria meningitidis, Neisseria spp., Nocardia spp., Pasteurella, Pasteurella multocida, Pasteurella spp., Pasteurella tularensis, Peptostreptococcus, Porphyromonas gingivalis, Prevotella melaninogenica (previously called Bacteroides melaninogenicus), Proteus spp., Pseudomonas aeruginosa, Pseudomonas spp., Rhizobium radiobacter, Rickettsia, Rickettsia prowazekii, Rickettsia psittaci, Rickettsia quintana, Rickettsia rickettsii, Rickettsia spp., Rickettsia trachomae, Rochalimaea, Rochalimaea henselae, Rochalimaea quintana, Rothia dentocariosa, Salmonella, Salmonella enteritidis, Salmonella spp., Salmonella typhi, Salmonella typhimurium, Serratia marcescens, Shigella dysenteriae, Shigella spp., Spirillum volutans, Staphylococcus, Staphylococcus aureus, Staphylococcus epidermidis, Staphylococcus spp., Stenotrophomonas maltophilia, Stenotrophomonas spp., Streptococcus, Streptococcus agalactiae, Streptococcus avium, Streptococcus bovis, Streptococcus cricetus, Streptococcus faceium, Streptococcus faecalis, Streptococcus feruns, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Streptococcus spp., Treponema, Treponema denticola, Treponema pallidum, Treponema spp., Ureaplasma spp., Vibrio, Vibrio cholerae, Vibrio comma, Vibrio parahaemolyticus, Vibrio spp., Vibrio vulnificus, Viridans streptococci, Wolbachia, Yersinia, Yersinia enterocolitica, Yersinia pestis, Yersinia pseudotuberculosis, and Yersinia spp.

The microorganism may be a fungus. Examples of fungi include, but are not limited to, Aspergillus spp., Blastomyces spp., Candida spp., Cladosporium, Coccidioides spp., Cryptococcus spp., Exserohilum, fusarium, Histoplasma spp., Issatchenkia spp., Mucormycetes, Pneumocystis spp., ringworm, Scedosporium, Sporothrix, and Stachybotrys spp. The microorganism may be a protozoan. Examples of protozoans include, but are not limited to, Entamoeba histolytica, Plasmodium spp., Giardia lamblia, and Trypanosoma brucei.

### Antimicrobials

When the microorganism is a bacterium, exemplary antimicrobials include Amikacin, Aminoglycoside, Aminoglycoside amoxicillin, Aminoglycosides, Amoxicillin, Amoxicillin/clavulanate, Ampicillin, Ampicillin/sulbactam, Antitoxin, Arsphenamine, Azithromycin, Azlocillin, Aztreonam, β-lactam, Bacitracin, Capreomycin, Carbapenems, Carbenicillin, Cefaclor, Cefadroxil, Cefalexin, Cefalothin, Cefalotin, Cefamandole, Cefazolin, Cefdinir, Cefditoren, Cefepime, Cefixime, Cefoperazone, Cefotaxime, Cefoxitin, Cefpodoxime, Cefprozil, Ceftaroline, Ceftaroline fosamil, Ceftazidime, Ceftibuten, Ceftizoxime, Ceftobiprole, Ceftriaxone, Cefuroxime, Cephalosporin, Chloramphenicol, Chloramphenicol(Bs), Ciprofloxacin, Clarithromycin, Clindamycin, Clofazimine, Cloxacillin, Colistin, Co-trimoxazole, Cycloserine, Dalbavancin, Dapsone, Daptomycin, Demeclocycline, Dicloxacillin, Dirithromycin, Doripenem, Doxycycline, Enoxacin, Ertapenem, Erythromycin, Ethambutol, Ethambutol(Bs), Ethionamide, Flucloxacillin, Fluoroquinolone, Fluoroquinolones, Fosfomycin, Furazolidone, Fusidic acid, Gatifloxacin, Geldanamycin, Gemifloxacin, Gentamicin, Grepafloxacin, Herbimycin, Imipenem/Cilastatin, Isoniazid, Kanamycin, Levofloxacin, Lincomycin, Linezolid, Lomefloxacin, Loracarbef, Macrolides, Mafenide, Meropenem, Methicillin, Metronidazole, Mezlocillin, Minocycline, Moxifloxacin, Mupirocin, Nafcillin, Nafcillin, Nalidixic acid, Neomycin, Netilmicin, Nitrofurantoin(Bs), Norfloxacin, Ofloxacin, Oritavancin, Oxacillin, Oxytetracycline, Paromomycin, Penicillin, Penicillin G, Penicillin V, Piperacillin, Piperacillin/tazobactam, Platensimycin, Polymyxin B, Posizolid, Pyrazinamide, Quinupristin/Dalfopristin, Radezolid, Raxibacumab, Rifabutin, Rifampicin, Rifampin, Rifapentine, Rifaximin, Roxithromycin, Silver sulfadiazine, Sparfloxacin, Spectinomycin, Spectinomycin(Bs), Spiramycin, Streptogramins, Streptomycin, Sulbactam, Sulfacetamide, Sulfadiazine, Sulfadimethoxine, Sulfamethizole, Sulfamethoxazole, Sulfanilimide, Sulfasalazine, Sulfisoxazole, Sulfonamidochrysoidine, Tedizolid, Teicoplanin, Teixobactin, Telavancin, Telithromycin, Temafloxacin, Temocillin, Tetracycline, Thiamphenicol, ticarcillin, Ticarcillin/clavulanate, Ticarcillin/clavulanic acid, Tigecycline, Tigecycline(Bs), Tinidazole, TMP/SMX, Tobramycin, Torezolid, Trimethoprim(Bs), Trimethoprim-Sulfamethoxazole, Troleandomycin, Trovafloxacin, Vancomycin, and generics thereof or a variant thereof.

Antimicrobials whose interactions with the microorganism affect and are affected by the negative charges on the microorganism surface can include: polycationic aminoglycosides, which upon binding the cell surface displace Mg′ ions, which bridge lipid membrane components, thereby disrupting the outer membrane and enhancing drug uptake; cationic polymyxins (colistin and polymyxin B), whose binding to the microorganism cell is also dependent on the membrane's negative charge and for which both mutational and plasmid-mediated resistance occurs by reducing membrane negative charge; and daptomycin, a lipopeptide that resembles host innate immune response cationic antimicrobial peptides and requires Ca′ and phosphatidyl glycerol for its membrane-disrupting mechanism of action and for which resistance can also involve alteration in cell surface charge.

When the microorganism is a fungus, exemplary antimicrobials include 5-fluorocytosine, Abafungin, Albaconazole, Allylamines, Amphotericin B, Ancobon, Anidulafungin, Azole, Balsam of Peru, Benzoic acid, Bifonazole, Butoconazole, Candicidin, Caspofungin, Ciclopirox, Clotrimazole, Cresemba, Crystal violet, Diflucan, Echinocandins, Econazole, Efinaconazole, Epoxiconazole, Fenticonazole, Filipin, Fluconazole, Flucytosine, Grifulvin V, Griseofulvin, Gris-Peg, Haloprogin, Hamycin, Imidazoles, Isavuconazole, isavuconazonium, Isoconazole, Itraconazole, Ketoconazole, Lamisil, Luliconazole, Micafungin, Miconazole, Natamycin, Noxafil, Nystatin, Omoconazole, Onmel, Oravig, Oxiconazole, Posaconazole, Propiconazole, Ravuconazole, Rimocidin, Sertaconazole, Sporanox, Sulconazole, Terbinafine, Terconazole, Thiazoles, Thiocarbamate antifungal, Tioconazole, Tolnaftate, Triazoles, Undecylenic acid, Vfend, Voriconazole, and generics thereof or a variant thereof.

When the microorganism is a protozoan, exemplary antimicrobials include 8-Aminoquinoline, Acetarsol, Agents against amoebozoa, Ailanthone, Amodiaquine, Amphotericin B, Amprolium, Antitrichomonal agent, Aplasmomycin, Arsthinol, Artelinic acid, Artemether, Artemether/lumefantrine, Artemisinin, Artemotil, Arterolane, Artesunate, Artesunate/amodiaquine, Atovaquone, Atovaquone/proguanil, Azanidazole, Azithromycin, Benznidazole, Broxyquinoline, Buparvaquone, Carbarsone, Carnidazole, Chiniofon, Chloroquine, Chlorproguanil, Chlorproguanil/dapsone, Chlorproguanil/dapsone/artesunate, Chlorquinaldol, Chromalveolate antiparasitics, Cinchona, Cipargamin, Clazuril, Clefamide, Clioquinol, Coccidiostat, Codinaeopsin, Cotrifazid, Cryptolepine, Cycloguanil, Dehydroemetine, Difetarsone, Dihydroartemisinin, Diloxanide, Diminazen, Disulfiram, Doxycycline, Eflornithine, ELQ-300, Emetine, Etofamide, Excavata antiparasitics, Fumagillin, Furazolidone, Glycobiarsol, GNF6702, Halofantrine, Hydroxychloroquine, Imidocarb, Ipronidazole, Jesuit's bark, KAF156, Lumefantrine, Maduramicin, Mefloquine, Megazol, Meglumine antimoniate, Melarsoprol, Mepacrine, Metronidazole, Miltefosine, Neurolenin B, Nicarbazin, Nifurtimox, Nimorazole, Nitarsone, Nitidine, Nitrofural, Olivacine, Ornidazole, Oroidin, Pamaquine, Paromomycin, Pentamidine, Pentavalent antimonial, Phanquinone, Phenamidine, Piperaquine, Primaquine, Proguanil, Project 523, Propenidazole, Pyrimethamine, Pyronaridine, Quinfamide, Quinine, Ronidazole, Schedula Romana, SCYX-7158, Secnidazole, Semapimod, Sodium stibogluconate, Spiroindolone, Sulfadoxine, Sulfadoxine-Pyrimethamine, Sulfalene, Suramin, Tafenoquine, Teclozan, Tenonitrozole, Tilbroquinol, Tinidazole, Trimetrexate, Trypanocidal agent, Warburg's tincture, and generics thereof or a variant thereof.

An antimicrobial may be a drug that operates by a mechanism similar to a herein-recited drug. Other antimicrobial drugs known in the art may be used in the methods described herein.

### Liquid Suspensions

The liquid may include a growth media, such as cation-adjusted Mueller Hinton broth. This media may comprise an additive, known to those skilled in the art to promote microorganism growth, and stability. In addition to different antimicrobials, different test wells may comprise an additive known to improve AST accuracy for specific antimicrobials. For example, additional sodium chloride may be added to tests comprising oxacillin and additional calcium may be added to tests comprising daptomycin.

### Biological Samples

The microorganisms described herein may be derived from biological samples. In some embodiments, the biological sample is any sample that comprises a microorganism, e.g., a bacterium and a fungal cell. The biological sample may be derived from a clinical sample.

Exemplary biological samples can include, but are not limited to, whole blood, plasma, serum, sputum, urine, stool, white blood cells, red blood cells, buffy coat, tears, mucus, saliva, semen, vaginal fluids, lymphatic fluid, amniotic fluid, spinal or cerebrospinal fluid, peritoneal effusions, pleural effusions, exudates, punctates, epithelial smears, biopsies, bone marrow samples, fluids from cysts or abscesses, synovial fluid, vitreous or aqueous humor, eye washes or aspirates, bronchoalveolar lavage, bronchial lavage, or pulmonary lavage, lung aspirates, and organs and tissues, including but not limited to, liver, spleen, kidney, lung, intestine, brain, heart, muscle, pancreas, and the like, swabs (including, without limitation, wound swabs, buccal swabs, throat swabs, nasal swabs, vaginal swabs, urethral swabs, cervical swabs, rectal swabs, lesion swabs, abscess swabs, nasopharyngeal swabs, and the like), and any combination thereof. Also included are bacteria cultures or bacteria isolates, fungal cultures or fungal isolates. The ordinary-skilled artisan may also appreciate that isolates, extracts, or materials obtained from any of the above exemplary biological samples are also within the scope of the present invention.

Microorganisms obtained from a biological sample may be cultured or otherwise processed as is routinely performed in the art.

### Controls Used in AST Methods

Controls may include antimicrobials for which the microorganism is not susceptible. As examples, if the assay is used to determine the susceptibility of gram-positive bacteria, then the controls (and the test incubations) may include one or more antimicrobials that target gram-negative bacteria, and if the assay is used to determine the susceptibility of eukaryotic microorganisms, the control (and the test incubations) may include one or more antibacterial antimicrobials.

In some embodiments, the control is a positive control measured from microorganisms under otherwise identical conditions but without antimicrobials or with one or more antimicrobials for which the microorganisms are not susceptible. In some embodiments, the control is measured from microorganisms under otherwise identical conditions but without nutrients. In some embodiments, the control is measured from microorganisms under otherwise identical conditions with one or more toxins known to inhibit growth of the microorganisms.

Controls may be historic controls. In some embodiments, the test incubations are performed after control incubations have been performed. In some embodiments, controls are performed in a cartridge distinct from the cartridge comprising the test incubations.

### Cartridges

A cartridge can be a container that is capable of holding and allowing growth of a liquid suspension of microorganisms. Non-limiting examples of a cartridge can include a culture flask, a culture dish, a petri dish, a bioassay dish, a culture tube, a test tube, a microfuge tube, a bottle, a microchamber plate, a multi-chamber plate, a microtiter plate, a microplate. The cartridge may comprise one chamber. The cartridge may include a plurality of chambers, each chamber being a space capable of holding a liquid suspension in physical isolation from another space; an example of a chamber is a chamber in a multiwall plate. The cartridge may comprise 1, 2, 3, 4, 5, 6, 7, 8, 9, 10, 11, 12, 24, 48, 96, 192, 384, 1536, or more chambers, and any number of chambers in between. The bottom of the cartridge chamber may be flat, round, or V-shaped.

Antimicrobials present within a plurality of chambers on the cartridge can be suspended in a medium. In some embodiments, the antimicrobial is present in the form of antimicrobial film. In certain embodiments, the antimicrobial is in solid form. In some embodiments, the solid antimicrobial is lyophilized and/or dried. Certain embodiments provide for one or more antimicrobials present in one or more cartridge chambers as antimicrobial films, in solid form, lyophilized, or dried prior to introduction of a suspension of microorganisms.

An antimicrobial dilution series may be frozen, lyophilized, or prepared fresh prior to plate inoculation with a sample. In some cases, inoculation of cartridges can be performed either by hand or using an automated system. In some examples, such as in cases of fresh antimicrobial plates, an automated liquid handling system may be used to prepare the cartridge with antimicrobial dilution series. Inoculation processes can include any of various processes that may be known in the art.

As described herein, cartridges can be used to contain various combinations of fluids in order to carry out multiple testing sequences, such as a check point assay and a plurality of different growth assays. In some embodiments, a cartridge has a set of chambers used to facilitate the one or more checkpoint assays and a set of chambers used to facilitate the one or more growth assays. By way of example, a cartridge can include an array of chambers arranged in rows and columns. The cartridge can include a set of control chambers and a set of antimicrobial testing chambers. The set of control chambers can include two chambers and the set of testing chambers can include the remainder of chambers along the plate. In some embodiments, the set of control chambers includes at least two chambers, where one chamber is a growth chamber and another chamber is a no-growth chamber. In some embodiments, the growth chamber includes, or be inoculated to include, a combination of broth and a patient sample such that the microorganisms in the patient sample can grow within the broth during an incubation period. In certain embodiments, antimicrobials are not added to the checkpoint assay chamber. Whereas, in some embodiments, the no-growth chamber can include, or be inoculated to include, broth without the patient sample (i.e., broth in the absence of the microorganisms from the patient sample). In some embodiments, antimicrobials are also not added to the no-growth chamber. Thus, during an incubation period, the no-growth chamber can serve as a baseline as compared to the growth chamber in which the microorganisms can grow.

In some embodiments, each cartridge includes a “test panel,” a plurality of antimicrobials distributed across multiple wells in a defined dilution series for each antimicrobial (e.g., a 2-fold dilution series, a 10-fold dilution series, etc.). In addition, each cartridge or test panel can contain control chambers, such as a growth control chamber, a no growth (contamination) control chamber and/or a saline control chamber. The saline control chamber can represent FIT control approximately equal to the initial concentration of microorganism in inoculum. The cartridges can include multiple chambers (e.g., 96 chamber cartridge or 384 chamber cartridge) with a cover (e.g., a removable lid) and an identifier (e.g., a bar code) that uniquely defines antibiotic configuration and a unique code, which defines the plate and can be associated with a unique patient sample conforming to HIPAA.

The testing chambers can include any of various combinations of the patient sample and various types and concentrations of antimicrobials for which susceptibility can be analyzed. Rows of chambers can be dedicated to a particular antimicrobial and concentrations of that antimicrobial can vary between columns of the same row. For example, a cartridge can have a row of chambers containing penicillin where each chamber from left to right contains an increasing concentration of penicillin.

Of course, other examples are possible. For example, the different chambers and sets of chambers can be positioned at any of various locations along a cartridge. Additionally, the different sets of chambers (e.g., control chambers and testing chambers) can include greater or fewer individual chambers along the cartridge. Additionally, in some cases, not all chambers are used/occupied during testing.

### Automated AST Methods

The methods described herein can be performed in an automated manner using commercially available equipment, custom made equipment, or a combination thereof. Automating the methods allows for performance of a greater number of assays as well as increased consistency among assays. Automation can also increase speed and resolution of these methods.

### Surface Binding Probe Assays

Surface-binding assays (also referred to as surface-binding probe assays) can utilize a signaling agent. Signaling agents typically comprise a moiety capable of binding to a microorganism (e.g., an antibody and/or a lectin that bind to a microorganism surface, a charged moiety and/or a functional moiety that non-specifically binds to the microorganism surface) and a chemical moiety capable of providing a signal or contributing to production of a signal (e.g., an enzyme chemiluminophore, and lanthanide chelate). Exemplary enzymes include horseradish peroxidase, alkaline phosphatase, acetyl cholinesterase, glucose oxidase, beta-D-galactosidase, beta-lactamase, and a combination thereof.

A signal generator may include one or more chemical moieties conjugated to one or more microorganism receptors. Signal generators include, but are not limited to, one or more catalysts (including enzymes, metal-oxide nanoparticles, organometallic catalysts, nanoparticles designed for signal amplification (such as those described in the U.S. Provisional applications to which the present application claims priority and incorporates by reference in their entireties), bacteriophages comprising signal generating elements, fluorophores (including organic fluorophores, europium, or ruthenium(II), rhenium(I), palladium(II), platinum(II)-containing organometallics), and/or colorimetric dyes (including organic stains). Combinations of the above may be used, such as nanoparticles, dendrimers, and/or other nanoscale structures with enzymes, fluorophores, and/or organometallic molecules.

The chemical moiety may be conjugated to a signaling agent before contacting the signaling agent to a microorganism, while the signaling agent is initially contacted to a microorganism, or after the signaling agent has contacted a microorganism.

When the signaling agents are added to AST dilutions containing a microorganism, signaling agent receptors (e.g., moieties that can bind specifically or non-specifically to a microorganism) may associate with microorganism surfaces. Thus, the more intact microorganisms, for example, there are in solution, the greater the number of signaling agents that will be associated with these bacteria. Consequently, there is an inverse relationship between the number of intact bacteria and the number of signaling agents that are free in solution, as defined by those not bound to intact bacteria. Note that free signaling agents may be bound to soluble microbial components if, for example, microorganisms lyse in response to antimicrobial treatment.

The number of signaling agents that associate with and/or intercalate into microorganism surfaces is proportional to the microorganism surface area. Microorganism surface area is strongly associated with truly resistant microorganisms. In particular, in the case of microorganisms that swell or elongate in response to MIC- and sub-MIC concentrations of antimicrobials (e.g., filament forming bacteria), metabolic and/or volumetric identifications are known to give false susceptibility profiles for rapid AST time points, defined as those less than six hours. To overcome this limitation, the present invention translates microorganism surface area (rather than volume) into a measurable signal such as an optical signal. The methods described herein are able to accurately determine microorganism resistance profiles in less than six hours.

In order to separate signaling agents associated with and/or intercalated into microorganisms from free signaling agents, it may be necessary to perform one or more separation and/or competitive binding steps. Such steps include, but are not limited to, centrifugation (e.g., with a g-force >500×g), filtration (e.g., via a filter having pores smaller than or equal to 0.45 microns, or smaller than or equal to 0.2 microns), electrophoresis, and/or magnetic capture; such steps are well-known to those skilled in the art.

In order to promote signaling agent binding and/or reduce background, it may further be advantageous, before adding signaling agents, to separate microorganisms from the liquid in which they were suspended during incubation. Such separations may include but are not limited to, centrifugation, filtration, electrophoresis, and/or magnetic capture.

Signaling agents may be added together with microorganisms and/or antimicrobials, such that they are present for the entire AST incubation period. This total period may be up to twenty-four hours, or within eight hours, or within five hours. Alternatively, signaling agents may be added to microorganisms and antimicrobial after a prescribed incubation period. This period may be up to twenty-four hours, or within eight hours, or within four hours.

Signaling agents are designed to associate with and/or intercalate in microorganism surfaces, including walls and/or membranes. Signaling agents designed for association comprise binding moieties including, but are not limited to, one or more antibodies, lectins, other proteins, small molecules with one or more charged chemical groups, small molecules with one or more functional chemical groups, phages, glycoproteins, peptides, aptamers, charged small molecules, small molecules with fixed charges, charged polymers, charged polymers with fixed charges, hydrophobic small molecules, charged peptide, charged peptides with fixed charges, peptides with alternating hydrophilic and hydrophobic regions, and/or small molecule ligands, which may or may not be organometallic complexes. Molecules designed for microorganism association are well-known to those skilled in the art. Signaling agents may remain bound to microorganisms and/or may be internalized, thus all associations are included. Signaling agents designed for intercalation may include, but are not limited to, small hydrophobic molecules, hydrophobic peptides, and/or peptides with alternating hydrophobic and hydrophilic regions. Molecules designed for microorganism intercalation are well-known to those skilled in the art. Signaling agents may further be specific to one or more types of microorganisms. Signaling agents may have multiple receptors. These may enhance binding and/or enable simultaneous binding to two or more microorganisms, which may further serve to agglutinate bacteria. Prior to or concurrently with the addition of signaling agents it may be advantageous to adjust the solution pH. This may be beneficial for enhancing charge-charge interactions between microorganisms and signaling agents. The anionic charge of microorganisms may be increased by titrating the solution pH above neutral (more basic). It may thus be beneficial to utilize moieties with one or more fixed, cationic charges.

It is noteworthy that the signaling agent may specifically bind to a microorganism (e.g., an antibody that specifically binds to a microorganism species or a strain of microorganism) or my non-specifically binds to a microorganism (e.g., by a generic covalent or non-covalent bond formation and another non-specific chemical association known in the art).

Alternately, chemicals and/or biochemicals which are capable of associating with signaling agents may be added to the liquid in which the microorganisms are suspended during growth, such that chemicals and/or biochemicals are incorporated into microorganisms during incubation. This may serve to enhance signaling agent association with microorganisms. In alternative embodiments, the signaling agents themselves may be present in the liquid in which the microorganisms are suspended during incubation and may be incorporated into microorganisms during growth.

The signaling agents can comprise an amplifier signal generator (amplifier group), such that the signal from each intact microorganism may be amplified beyond the number of signaling agents associated with each microorganism. For example, the enzyme horseradish peroxidase (HRP) is known to be able to amplify signals >1×10⁴-fold. Thus, if one hundred HRP molecules are bound to each microorganism surface, an amplification of 10⁶ may be achieved. This may increase the speed with which AST determinations may be made by enabling discrimination of microorganism concentrations that cannot otherwise be differentiated. Use of Europium formulations similarly provides signal amplification.

Alternatively, the signaling agents may comprise optical dye precursors known to those skilled in the art as membrane dyes that are designed to greatly increase fluorescence emission upon intercalation into a hydrophobic region, such as a cell membrane. Assays designed with these signaling agents may require microorganisms to be concentrated into a smaller volume, approaching a plane, to produce sufficient signals so as to be easily optically measured. Interfering species may require the use of near-IR fluorophores.

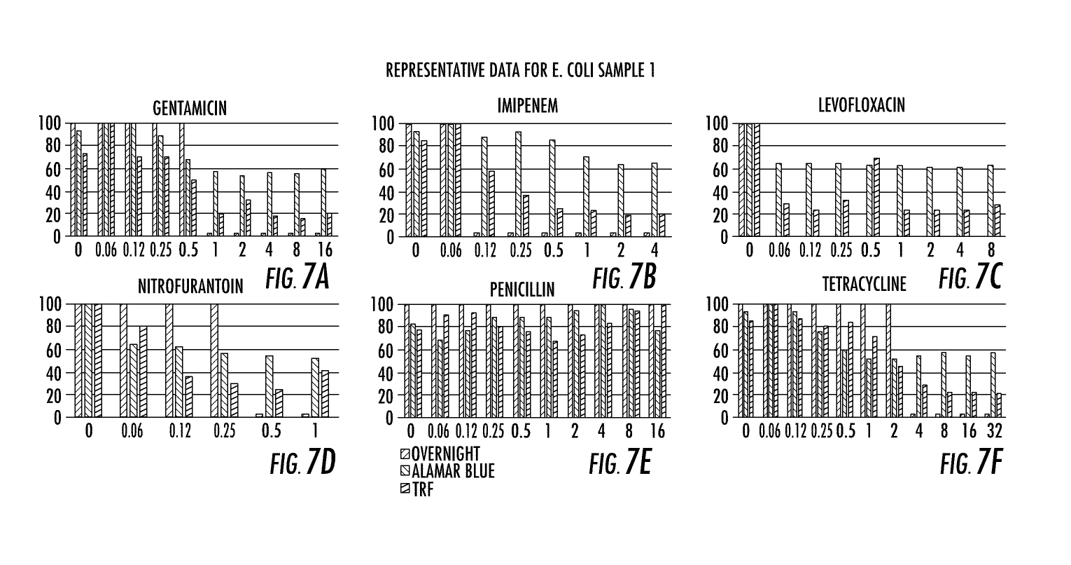

Exemplary amplifier groups include those described in, e.g., International Publication No. WO 2016/015027 and in International Application No. PCT/US16/42589, each of which is incorporated by reference in its entirety. An amplifier group can comprise a catalyst, a fluorophore, a colormetric dye, an enzyme, a catalyst, or a nanoparticle. Exemplary fluorophores include those described in FIG. 7, Table 1 of International Application No. PCT/US16/42589, which is incorporated by reference in its entirety. An amplifier group can comprise a lanthanide. Lanthanides include, but are not limited to, is europium, strontium, terbium, samarium, or dysprosium.

An amplifier group can comprise an organic fluorophore, e.g., a coordination complex. The coordination complex can be europium coordination complex, a ruthenium coordination complex, a rhenium coordination complex, a palladium coordination complex, a platinum coordination complex. An amplifier can comprise a chemiluminophore, a quantum dot, an enzyme, an iron coordination catalyst, a europium coordination complex, a ruthenium coordination complex, a rhenium coordination complex, a palladium coordination complex, a platinum coordination complex, a samarium coordination complex, a terbium coordination complex, or a dysprosium coordination complex.

In some embodiments, an amplifier group comprises a moiety that is:

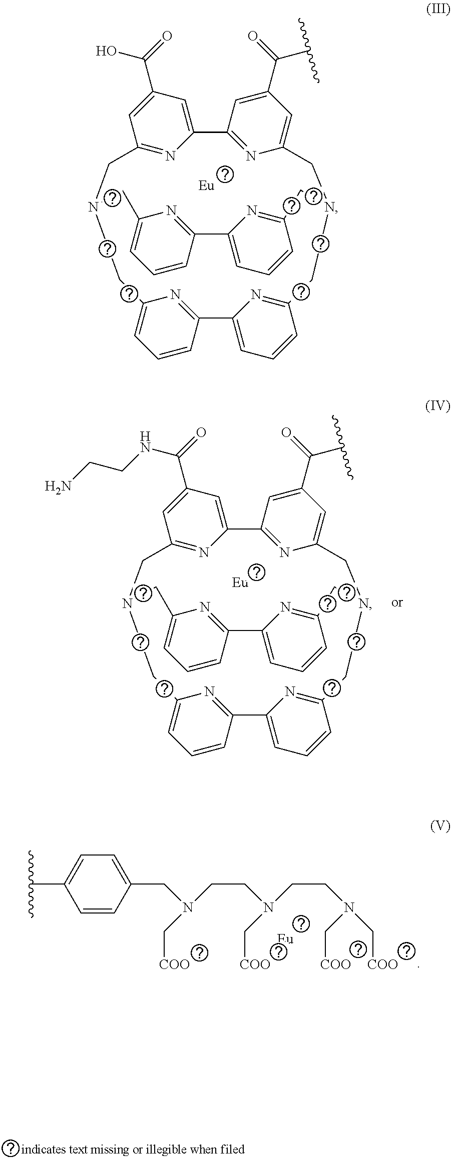

In some embodiments, an amplifier group comprises a moiety that is:

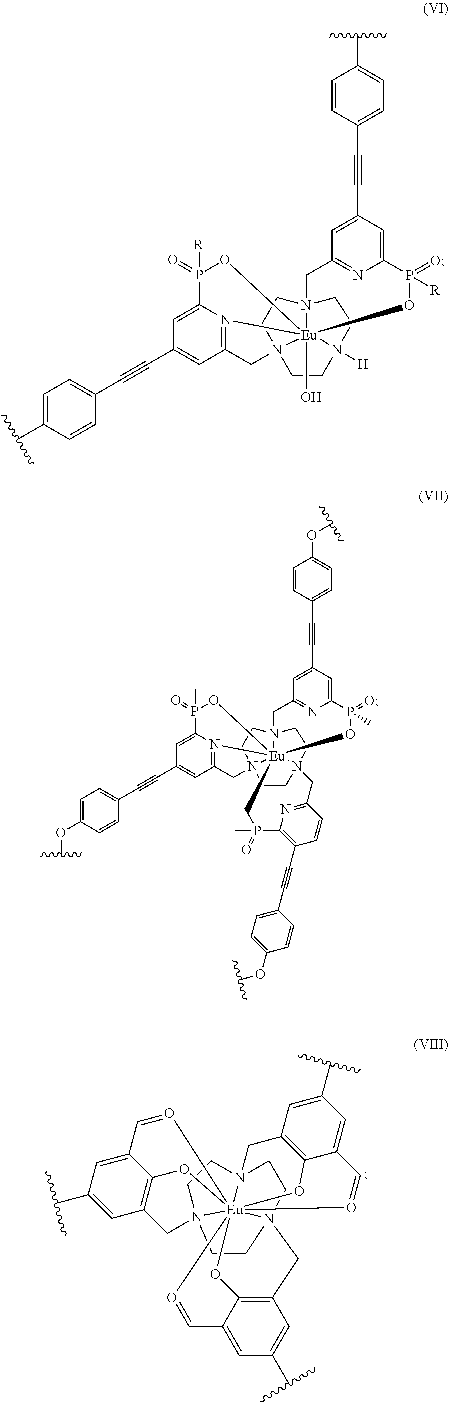

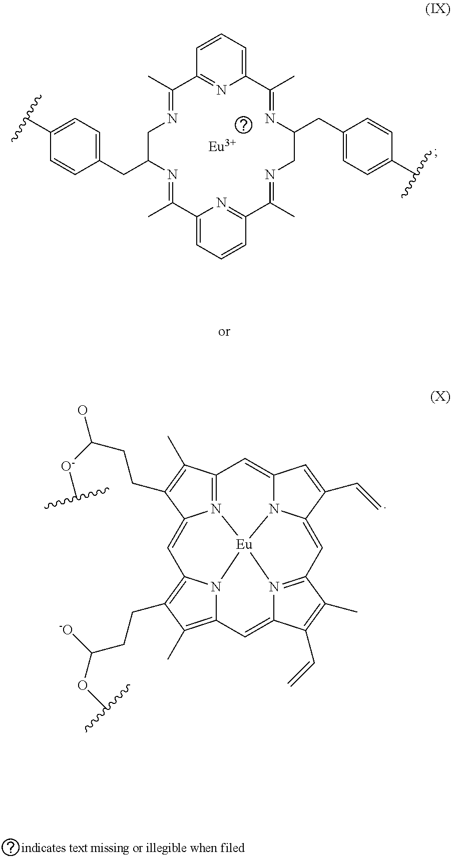

An amplifier group can comprise a fluorophore or colormetric dye. Suitable fluorophores and colormetric dyes are well known to those skilled in the art and are described in The Molecular Probes® Handbook: A Guide to Fluorescent Probes and Labeling Technologies, 11^{th} Ed. (2010) and Gomes, Fernandes, and Lima J. Biochem. Biophys. Methods 65 (2005) pp 45-80, which are herein incorporated by reference in their entirety. Exemplary fluorophores also include those described in, e.g., International Publication No. WO 2016/015027 and in International Application No. PCT/US16/42589, each of which is incorporated by reference in its entirety.

Examples of suitable fluorophore or colormetric dyes include, but are not limited to, ethidium bromide, propidium iodide, SYTOX green, phenanthridines, acridines, indoles, imidazoles, cyanine, TOTO, TO-PRO, SYTO, 5-carboxy-2,7-dichlorofluorescein, 5-Carboxyfluorescein (5-FAM), 5-Carboxynapthofluorescein, 5-Carboxytetramethylrhodamine (5-TAMRA), 5-FAM (5-Carboxyfluorescein), 5-HAT (Hydroxy Tryptamine), 5-ROX (carboxy-X-rhodamine), 6-Carboxyrhodamine 6G, 7-Amino-4-methylcoumarin, 7-Aminoactinomycin D (7-AAD), 7-Hydroxy-4-methylcoumarin, 9-Amino-6-chloro-2-methoxyacridine, ACMA (9-Amino-6-chloro-2-methoxyacridine), Acridines, Alexa Fluors, Alizarin, Allophycocyanin (APC), AMCA (Aminomethylcoumarin), Bodipy, Carboxy-X-rhodamine, Catecholamine, Fluorescein (FITC), Hydroxycoumarin, Lissamine Rhodamine, Monobromobimane, Oregon Green, Phycoerythrin, SYTO, Thiadicarbocyanine (DiSC3), Thioflavin, X-Rhodamine, C or TetramethylRodaminelsoThioCyanate.

An amplifier group can comprise an organometallic compound, transition metal complex, or coordination complex. Examples of such amplifier groups include, but are not limited to, those described in EP 0 180 492, EP 0 321 353, EP 0 539 435, EP 0 539 477, EP 0 569 496, EP139675, EP64484, U.S. Pat. Nos. 4,283,382, 4,565,790, 4,719,182, 4,735,907, 4,808,541, 4,927,923, 5,162,508, 5,220,012, 5,324,825, 5,346,996, 5,373,093, 5,432,101, 5,457,185, 5,512,493, 5,527,684, 5,534,622, 5,627,074, 5,696,240, 6,100,394, 6,340,744, 6,524,727, 6,717,354, 7,067,320, 7,364,597, 7,393,599, 7,456,023, 7,465,747, 7,625,930, 7,854,919, 7,910,088, 7,955,859, 7,968,904, 8,007,926, 8,012,609, 8,017,254, 8,018,145, 8,048,659, 8,067,100, 8,129,897, 8,174,001, 8,183,586, 8,193,174, 8,221,719, 8,288,763, 8,362,691, 8,383,249, 8,492,783, 8,632,753, 8,663,603, 8,722,881, 8,754,206, 8,890,402, 8,969,862, 9,012,034, 9,056,138, 9,118,028, 9,133,205, 9,187,690, 9,193,746, 9,312,496, 9,337,432, 9,343,685, 9,391,288, and 9,537,107, which are incorporated by reference in their entirety. Exemplary organometallic compounds, transition metal complexes, or coordination complexes also include those described in, e.g., International Publication No. WO 2016/015027 and in International Application No. PCT/US16/42589, each of which is incorporated by reference in its entirety.

In some embodiments, amplifier group is a lanthanide coordination complex such as a complex between a lanthanide (e.g., Eu or Tb) and a tetradentate ligand or a complex between a lanthanide (e.g., Eu or Tb) and a cryptate ligand. In some embodiments, amplifier group is a coordination complex of Lanthanum (La), Cerium (Ce), Praseodymium (Pr), Neodymium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu), Ruthenium (Ru), Rhodium (Rh), Palladium (Pd), Osmium (Os), Iridium (Ir), or Platinum (Pt). In some embodiments, amplifier group is a coordination complex of a rare earth metal collectively refers to 17 elements consisting of a group of 15 elements from lanthanum having an atomic number of 57 to lutetium having an atomic number of 71 (lanthanides), and two additional elements consisting of scandium having an atomic number of 21 and yttrium having an atomic number of 39. Specific examples of rare earth metals include europium, terbium, lanthanum, cerium, praseodymium, neodymium, promethium, samarium, gadolinium, dysprosium, holmium, erbium, thulium, ytterbium, lutetium, scandium and yttrium. In some embodiments, amplifier group is a coordination complex of a lanthanide (e.g., Europium or Terbium) with diethylenetriaminetetraacetic acid or cryptate ligand.

Specific examples of a signaling agent include, but are not limited to, moieties comprising:

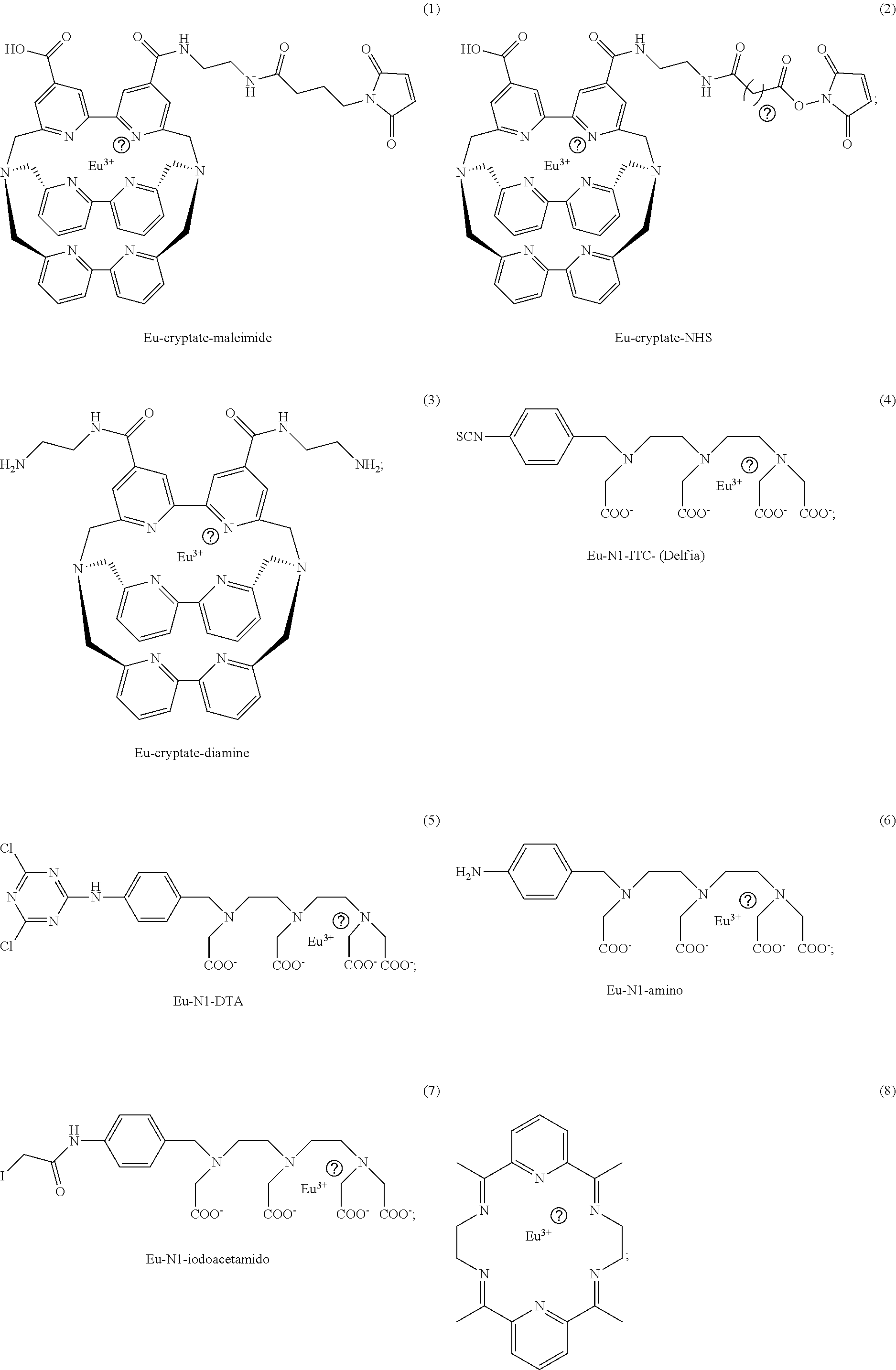

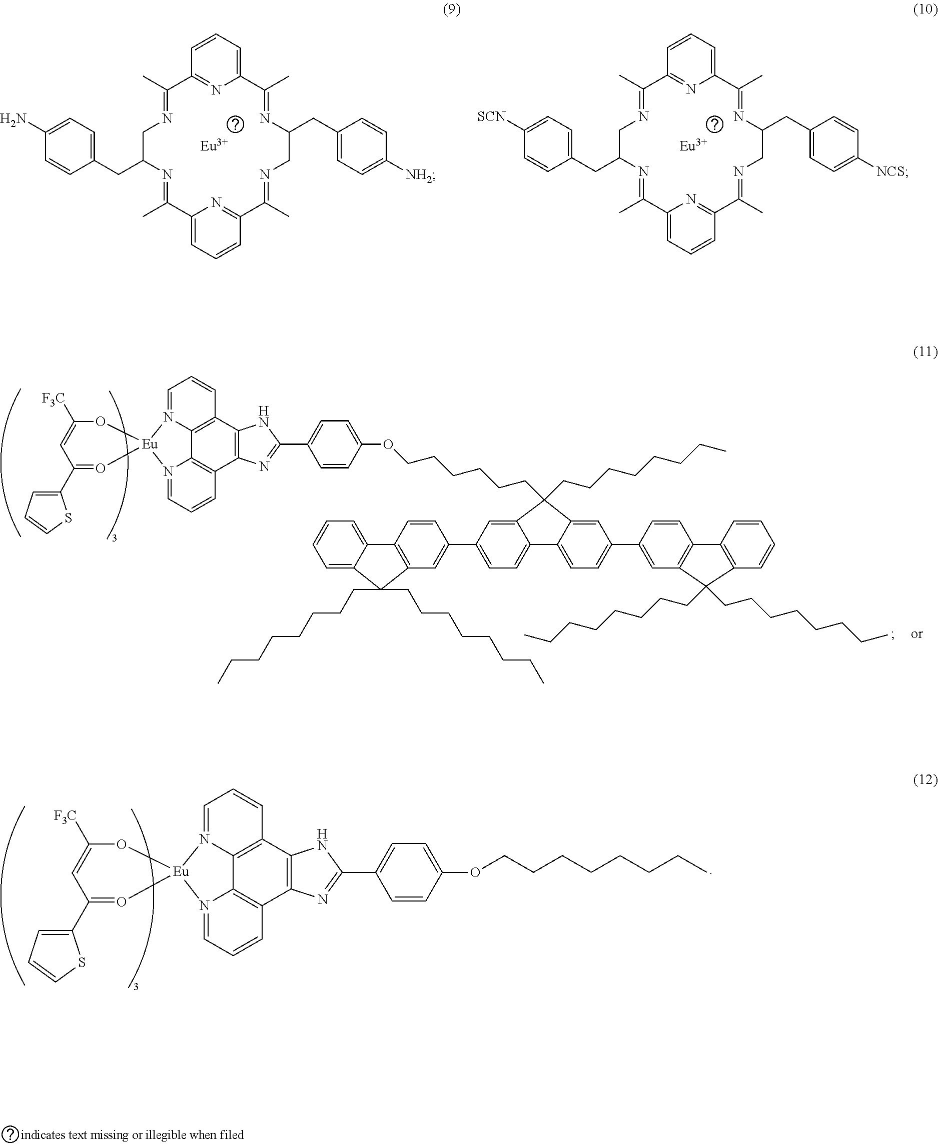

A signaling agent can comprise a luminophore (donor) which features high luminescence quantum efficiency and long luminescence decay time (>100 ns). Exemplary luminophores are cationic, metalorganic complexes of palladium, rhodium, platinum, ruthenium, osmium, rare earths (in particular, europium and lanthanum). The organic portion of these metalorganic complexes may consist, for example, of ligands from the group of porphyrins, bipyridyls, phenanthrolines or other heterocyclical compounds.

In some embodiments, a signaling agent capable of binding a microorganism surface comprises an antibody (e.g., monoclonal or polyclonal), modified antibodies (e.g., biotinylated monoclonal antibody, biotinylated polyclonal antibody, europium chelate-antibody, horseradish peroxidase-conjugated antibody), antibody variants (e.g., Fab: fragment, antigen-binding (one arm); F(ab′)2: fragment, antigen-binding, including hinge region (both arms); Fab′: fragment, antigen-binding, including hinge region (one arm); scFv: single-chain variable fragment; di-scFv: dimeric single-chain variable fragment; sdAb: single-domain antibody; Bispecific monoclonal antibodies; trifunctional antibody; and BiTE: bi-specific T-cell engager), WGA-Biotin, PolymixinB-Biotin, lectin, natural peptide, synthetic peptides, synthetic and/or natural ligands, synthetic and/or natural polymers, synthetic and/or natural glycopolymers, carbohydrate-binding proteins and/or polymers, glycoprotein-binding proteins and/or polymers, charged small molecules, other proteins, bacteriophages, and/or aptamers.

In some embodiments, a signaling agent capable of binding a microorganism surface comprises or is formed from a structure comprising an antibody, lectin, natural peptide, synthetic peptides, synthetic and/or natural ligands, synthetic and/or natural polymers, synthetic and/or natural glycopolymers, carbohydrate-binding proteins and/or polymers, glycoprotein-binding proteins and/or polymers, charged small molecules, other proteins, bacteriophages, and/or aptamers.

In some embodiments, a signaling agent capable of binding a microorganism surface comprises an amplifier group that comprises a lanthanide coordination complex, and/or an enzyme and streptavidin and/or an antibody and/or aptamer. In some embodiments, a signaling agent capable of binding a microorganism surface comprises a binding moiety comprising a polyclonal and/or monoclonal antibody.

In some embodiments, a signaling agent capable of binding a microorganism surface comprises a binding moiety comprising a modified antibody. Exemplary modified antibodies include a biotinylated monoclonal antibody, biotinylated polyclonal antibody, a europium chelate-antibody, and a horseradish peroxidase-conjugated antibody. In some embodiments, a signaling agent capable of binding a microorganism surface comprises a binding moiety comprising an antibody variant. Exemplary antibody variants include Fab: fragment, antigen-binding (one arm); F(ab′)2: fragment, antigen-binding, including hinge region (both arms); Fab′: fragment, antigen-binding, including hinge region (one arm); scFv: single-chain variable fragment; di-scFv: dimeric single-chain variable fragment; sdAb: single-domain antibody; Bispecific monoclonal antibodies; trifunctional antibody; and BiTE: bi-specific T-cell engager),

In some embodiments, a signaling agent capable of binding a microorganism surface comprises WGA-Biotin or PolymixinB-Biotin. In some embodiments, a signaling agent capable of binding a microorganism surface comprises a binding moiety comprising a synthetic and/or natural ligand and/or peptide. In some embodiments, a ligand and/or peptide is selected from bis(zinc-dipicolylamine), TAT peptide, serine proteases, cathelicidins, cationic dextrins, cationic cyclodextrins, salicylic acid, lysine, and combinations thereof. In some embodiments, a signaling agent capable of binding a microorganism surface comprises a binding moiety comprising a synthetic and/or natural polymer and/or glycopolymer. In embodiments, a natural and/or synthetic polymer is linear or branched and selected from amylopectin, Poly(N-[3-(dimethylamino)propyl] methacrylamide), poly(ethyleneimine), poly-L-lysine, poly[2-(N,N-dimethylamino)ethyl methacrylate], and combinations thereof. In some embodiments, a natural and/or synthetic polymer and/or glycopolymer comprises moieties including, but not limited to, chitosan, gelatin, dextran, trehalose, cellulose, mannose, cationic dextrans and cyclodextrans, quaternary amines, pyridinium tribromides, histidine, lysine, cysteine, arginine, sulfoniums, phosphoniums, or combinations thereof including, but not limited to, co-block, graft, and alternating polymers. In some embodiments, a signaling agent capable of binding a microorganism surface comprises a binding moiety comprising a glycoprotein selected from mannose-binding lectin, other lectins, annexins, and combinations thereof.

In some embodiments, a signaling agent capable of binding to a microorganism surface comprises: an antibody; and a europium coordination complex. In some embodiments, a signaling agent capable of binding to a microorganism surface comprises a linker group L that comprises NH₂-PEG-Biotin (2K), NH₂-PEG-Biotin (4K), sulfo-NHS-Biotin, WGA-Biotin, or polymixinB-Biotin. In some embodiments, a signaling agent capable of binding to a microorganism surface comprises a europium complex comprises:

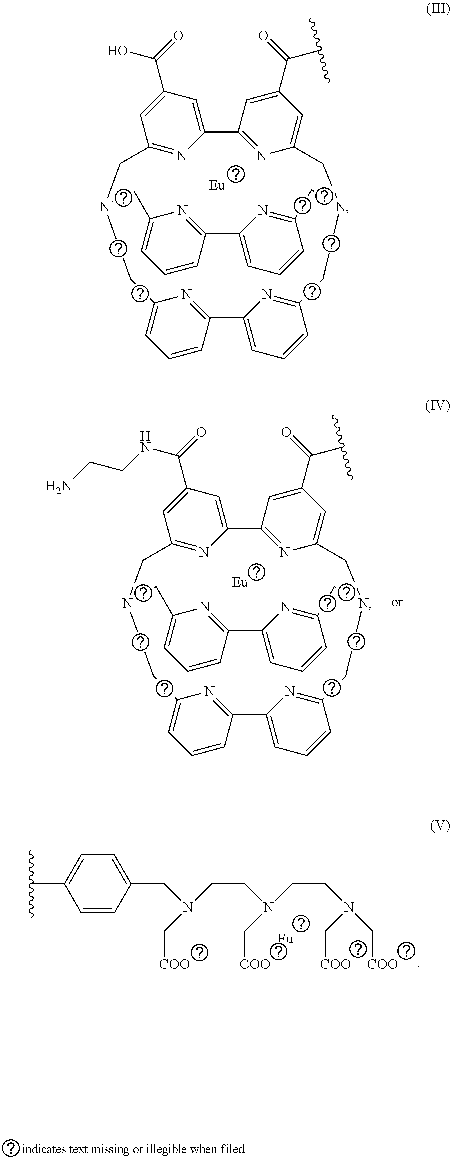

In some embodiments, a signaling agent capable of binding to a microorganism surface comprises a europium complex comprises:

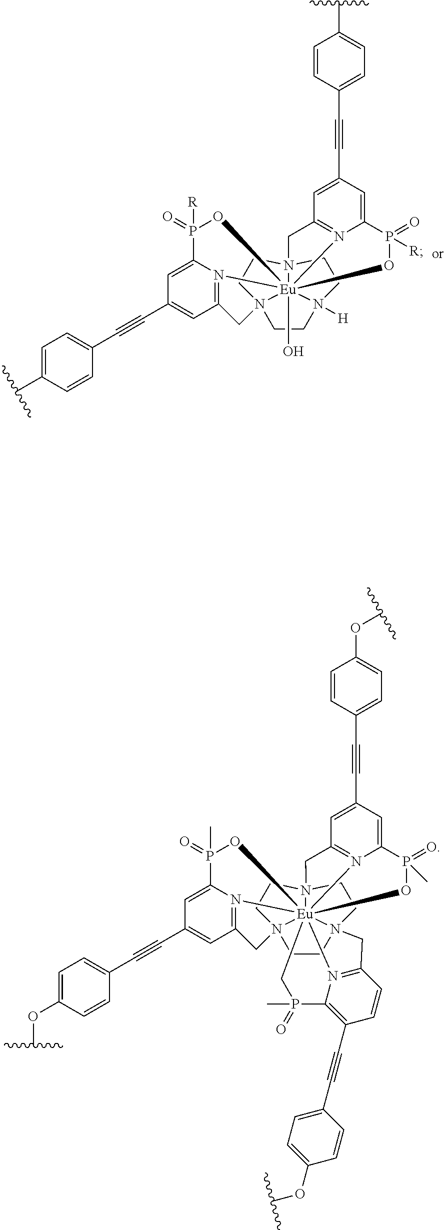

Alternatively, the signaling agents may be part of a pair, such as FRET/TR-FRET donor and acceptors or singlet oxygen pairs consisting of a photosensitizer and detector. Assays designed with these signaling agents may require the separation of the microorganisms from the initial growth media, with subsequent resuspension into a desired reaction buffer prior to the addition of the signaling reagents. Conversely, assays designed with these signaling agents may require no separation steps due to the required relative distance necessary to generate a signal.

Examples of FRET/TR-FRET donors include, but are not limited to, Lanthanide (Eu, Sm, Dy, or Tb)-containing cryptate organometallic (CisBio), Lance Eu-W1024 (Perkin Elmer), Lance Eu-W8044 (Perkin Elmer), also any organic fluorescent pair donor.

Examples of FRET/TR-FRET acceptors include, but are not limited to, matched organic dyes, such as ULight dye (Perkin Elmer), SureLight APC (Perkin Elmer), allophycocyanin, Cy5, d2 dye (CisBio), also any organic fluorescent pair acceptor.

Examples of singlet oxygen photosensitizers include, but are not limited to, methuselah Green Carboxy (Ursa Bio), Sensitizer Blue (Ursa Bio), rose Bengal, Erythrosin B, methylene blue, chlorophylls, AlphaBead donor (Perkin Elmer).

Examples of singlet oxygen detectors include, but are not limited to, singlet oxygen detector green (ThermoFisher), trans-1-(2′-methoxyvinyl)pyrene, Si-DMA (Dojindo), AlphaBead acceptor (Perkin Elmer).

Examples of incorporators include, but are not limited to, ethynyl-D-alanine (EDA), azido-D-alanine (ADA), fluorescent D-alanines described in Angew Chem Int Ed Engl. 2012 Dec. 7; 51(50): 12519-12523.

### EXAMPLES

### Example 1

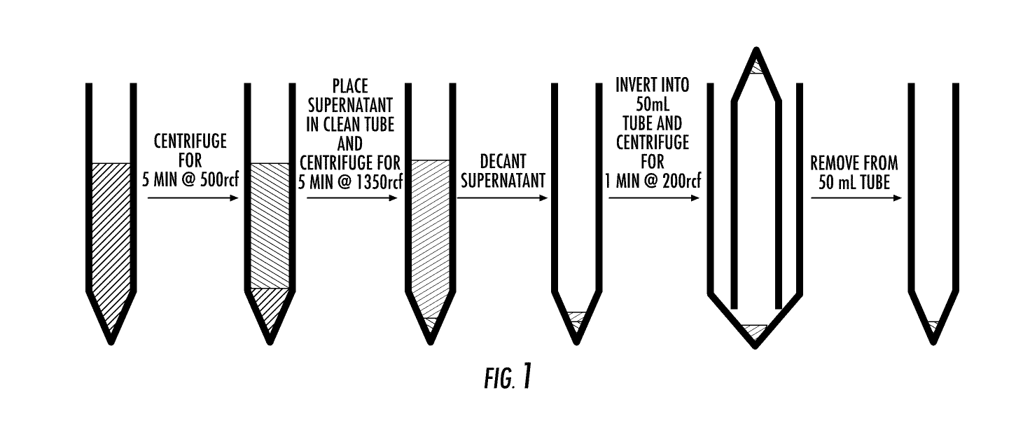

Human Blood (5 mL) and bacteria were added to 20 mL BACTEC growth media to obtain a bacteria load of 1000 cfu/mL. 12 mL of the bacteria solution was placed into a 15 mL falcon tube. The tube was spun at 500 RCF for 5 minutes. The resulting supernatant was removed and placed into a clean 15 mL tube. The tube containing the supernatant was spun at 1350 RCF for 5 minutes, producing a pellet of bacteria. The supernatant was removed and the 15 mL tube inverted into a 50 mL tube. The tubes were spun in this position at 200 RCF for 1 minute. The resulting bacteria pellet was resuspended into 500 uL of MHB. 3 150 uL aliquots were placed into a 96 well plate and growth monitored optically. After 6 hours, the sample was diluted 10 fold and inoculated across an AST plate. AST data was read after 20 hours. This process is illustrated in FIG. 1.

### Example 2

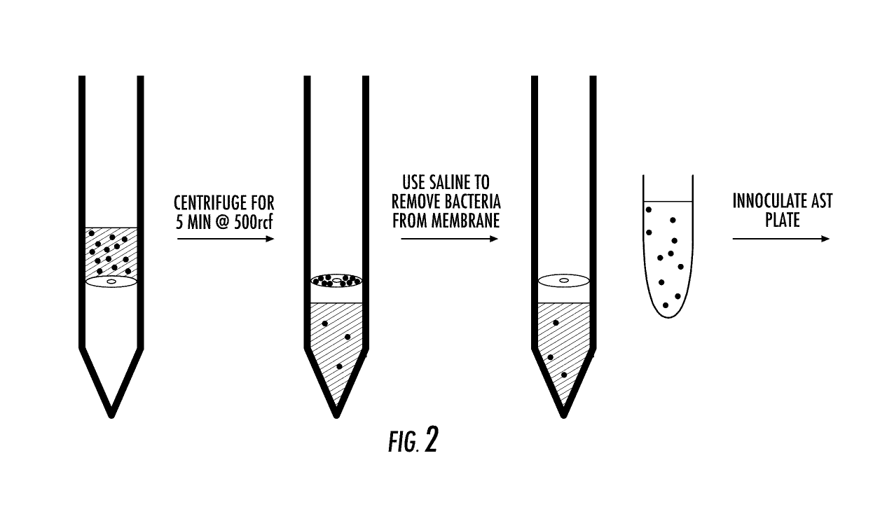

A urine sample (2 mL) was placed into two 10 mL LeucoSep centrifuge tubes. The tubes were spun at 500 RCF for 5 minutes. 2 mL of saline was added to the tubes to resuspend the bacteria. 2 mL was added a second time to wash the membrane. The contents of the tubes were combined and measured optically. The sample was diluted and inoculated across two AST plates. The plates were read using the SeLux assay and the CLSI guidelines. This process is illustrated in FIG. 2.

### Example 3

A positive blood culture (PBC) was removed from its bottle. One column of a 96-well plate was filled with 90 μL of saline and then 10 μL PBC was added. Concurrently, 10 μL of 10⁻³ through 10⁻⁸ dilutions were spot plated onto an agar plate and incubated overnight. 4 mL of PBC was added to 2 5 mL Sarstedt Screw Cap tubes (2 tubes per PBC per lysis detergent). The tubes were centrifuged at 500 RCF for 2 minutes. 3 mL supernatant was moved to clean tubes where 1 mL lysis detergent (3% Saponin, 1.53% Sodium Polyanethole Sulfonate, 0.000008% Polypropylene Glycol [4000 Mₙ]) was added and the tubes were vortexed. The tubes were centrifuged at 2370 RCF for 5 min. The supernatant was aspirated, 1 mL saline added, and the tubes vortexed. The tubes were again centrifuged at 2370 RCF for 5 min. The supernatant was again aspirated, 1 mL saline added, and the tubes vortexed, then combined into 1 tube. The tubes were centrifuged at 2370 RCF for 5 min. The supernatant was aspirated, 1 mL saline added, and the tubes vortexed. The tubes were centrifuged at 2370 RCF for 5 min. The supernatant was aspirated, 1 mL saline added, and the tubes vortexed. The resuspension was moved to a cuvette and the optical density was taken. One column of a 96-well plate was filled with 90 uL of saline. 10 μL of resuspension was added to the first wells of the row and then 1:10 dilutions were made down until dilutions from 10⁻¹ to 10⁻⁸ were prepared. 10 μL of 10⁻³ through 10⁻⁸ dilutions were spot plated onto an agar plate and incubated overnight.

### Example 4

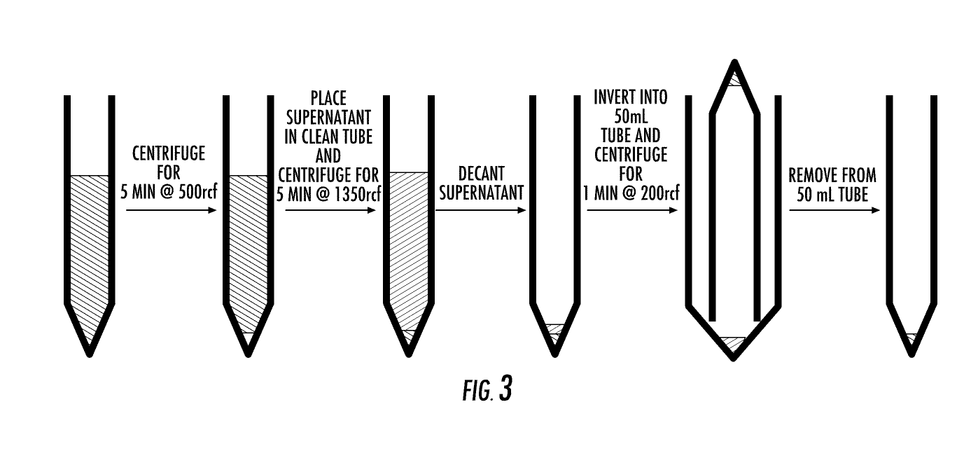

5 mL of a bacteria laden urine sample is placed into a 15 mL falcon tube. The tube is spun at 500 RCF for 5 minutes. The supernatant from this initial spin is removed and placed into a clean 15 mL tube. The supernatant containing tube is spun at 1350 RCF for 5 minutes to pellet the bacteria. The majority of the supernatant is decanted off before inverting the 15 mL tube into a 50 mL tube which is then spun at 200 RCF for 1 minute. The resulting bacteria pellet is then resuspended into 5 mL of saline and the bacteria concentration is determined optically. The sample is diluted to give the appropriate concentration for AST and is inoculated across two separate AST plates. One plate is processed using the SeLux assay and another is placed in an incubator overnight to be read according the CLSI guidelines. This process is illustrated in FIG. 3.

### Example 5

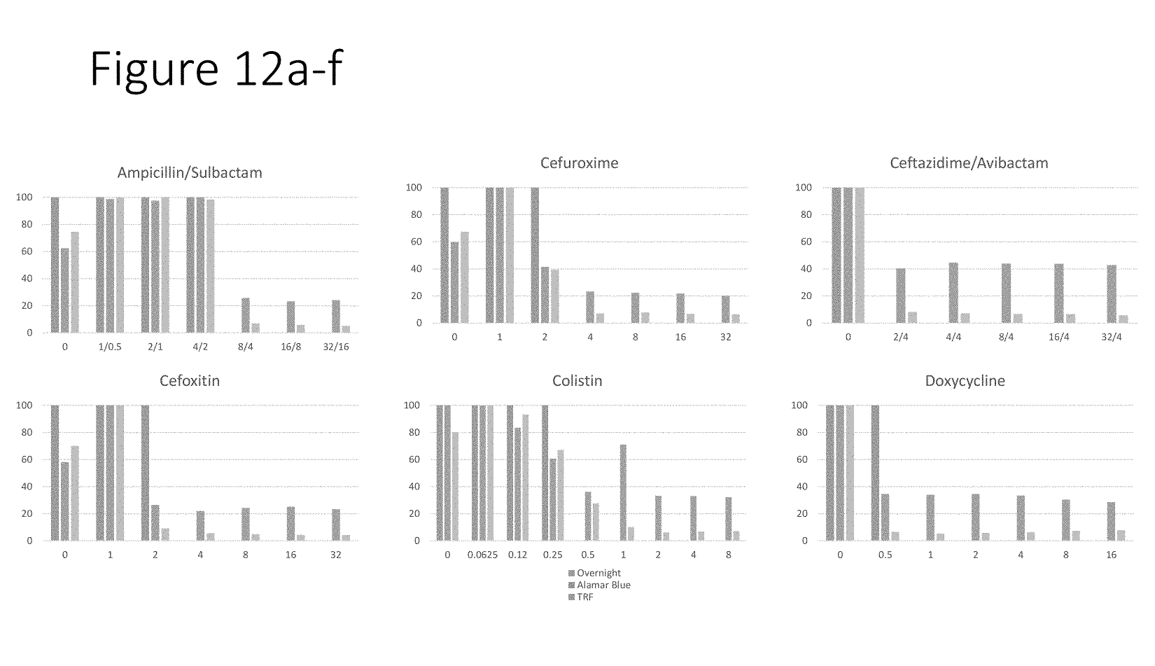

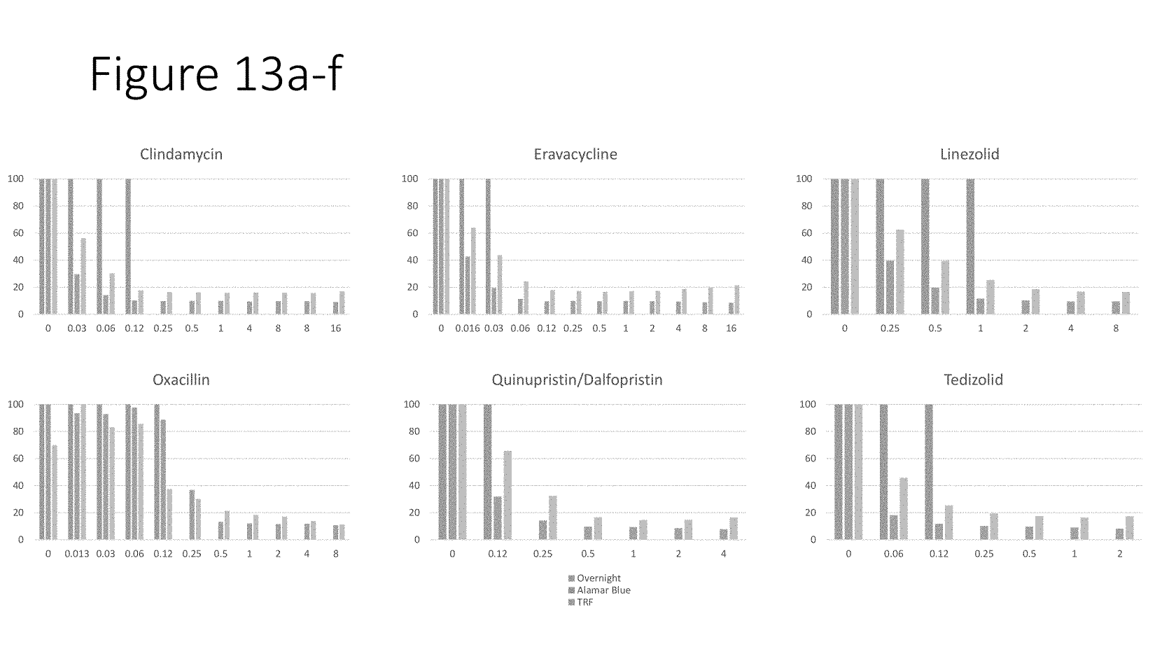

The positive blood culture (PBC) is removed from the blood culture bottle. One column (per PBC) of a 96-well plate is filled with 90 μL of saline. 10 μL of PBC is added to the first wells of the rows and then 1:10 dilutions are made down until dilutions from 10⁻¹ to 10⁻⁸ are prepared. 10 μL of 10⁻³ through 10⁻⁸ dilutions are spot plated onto an agar plate and incubated overnight. 4 mL of PBC is added to 2, 5 mL Sarstedt Screw Cap tubes (2 tubes per PBC per lysis detergent). Tubes are centrifuged at 500 RCF for 2 minutes. 3 mL supernatant per tube is moved to clean tubes. 1 mL Lysis Detergent is added, and tubes are vortexed (Detergent 12: 3% Saponin, 1.53% Sodium Polyanethole Sulfonate, 8×10⁻⁶% Polypropylene Glycol [4000 Mn]). Tubes are centrifuged at 2370 RCF for 5 minutes. Supernatant is aspirated, 1 mL saline is added, and tubes are vortexed. Tubes are centrifuged at 2370 RCF for 5 minutes. Supernatant is aspirated, pellets are washed with 1 mL saline again, and tubes are vortexed and then combined into 1 final tube. Tubes are centrifuged at 2370 RCF for 5 minutes. Saline is aspirated, and 1 mL of saline is added, and tubes are vortexed. Tubes are centrifuged at 2370 RCF for 5 minutes. Saline is aspirated, and 1 mL of saline is added, and tubes are vortexed. Resuspension is moved to a cuvette and the OD is taken (1st blanked with saline). One column (per final resuspension) of a 96-well plate is filled with 90 μL of saline. 10 μL of resuspension is added to the first wells of the row and then 1:10 dilutions are made down until dilutions from 10⁻¹ to 10⁻⁸ are prepared. 10 μL of 10⁻³ through 10⁻⁸ dilutions are spot plated onto an agar plate and incubated overnight. This process is illustrated in FIG. 5 with representative data shown in FIGS. 12-13

### Example 6

2 mL of blood from a BacTec bottle was added to a 5 mL tube. Centrifuged between 100-500 RCF over 1-5 minutes. Pulled off 1 mL of supernatant to check OD 600 after centrifugation. Spectrophotometer was blanked with supernatant from a 2 mL tube that was centrifuged at 2370 RCF (max speed) for 10 minutes. This process is illustrated in FIG. 3.

### Example 7

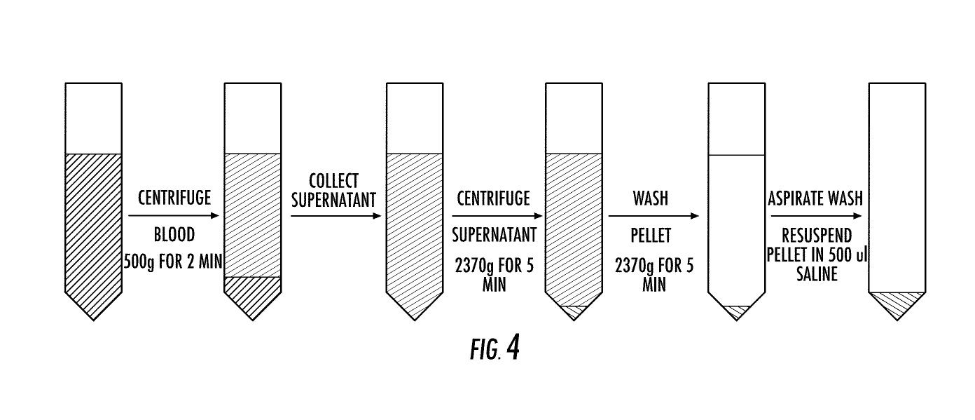

The positive blood culture (PBC) was removed from the blood culture machine. 4 mL of PBC was added to 2, 5 mL Sarstedt Screw Cap tubes (2 tubes per PBC per lysis detergent). Tubes were centrifuged at 500 RCF for 2 minutes. 3 mL supernatant per tube was moved to clean tubes. 1 mL Lysis Detergent was added, and tubes were vortexed (Detergent 12: 3% Saponin, 1.53% Sodium Polyanethole Sulfonate, 8×10⁻⁶% Polypropylene Glycol [4000 Mn]). Tubes were centrifuged at 2370 RCF for 5 minutes. Supernatant was aspirated, 1 mL saline was added, and tubes were vortexed. Tubes were centrifuged at 2370 RCF for 5 minutes. Supernatant was aspirated, pellets were washed with 1 mL saline again, and tubes were vortexed and then combined into 1 final tube. Tubes were centrifuged at 2370 RCF for 5 minutes. Saline was aspirated, and 1 mL of saline was added, and tubes were vortexed. Tubes were centrifuged at 2370 RCF for 5 minutes. Saline was aspirated, and 1 mL of saline was added, and tubes were vortexed. Resuspension was moved to a cuvette and the OD was taken (1st blanked with saline). One column (per final resuspension) of a 96-well plate was filled with 90 μL of saline. 10 μL of resuspension was added to the first wells of the row. This process is illustrated in FIG. 4.

### Example 8

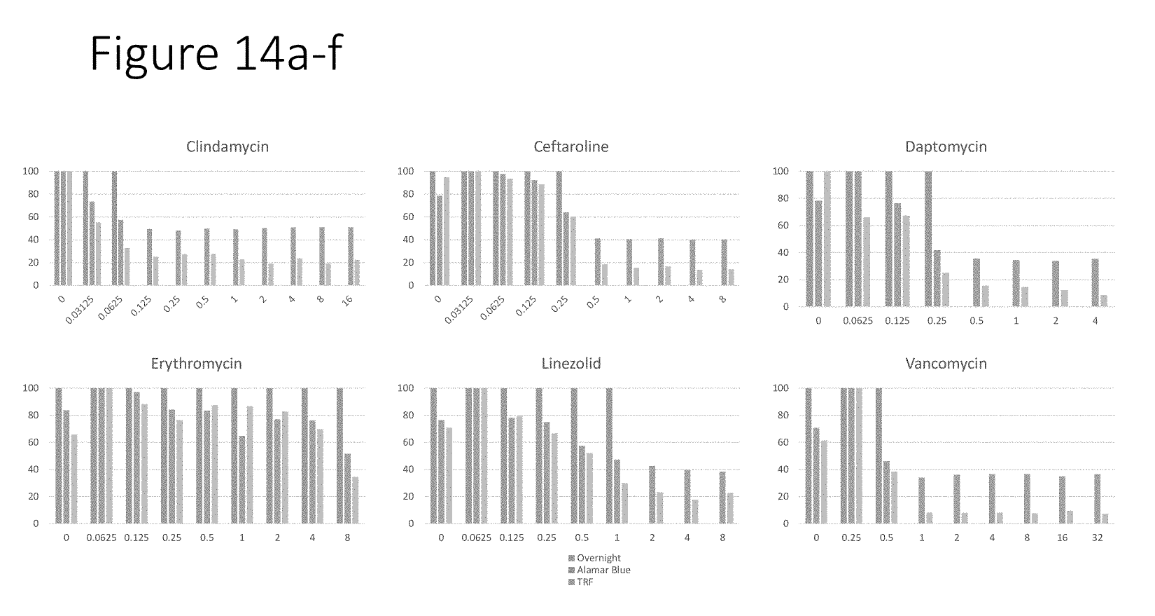

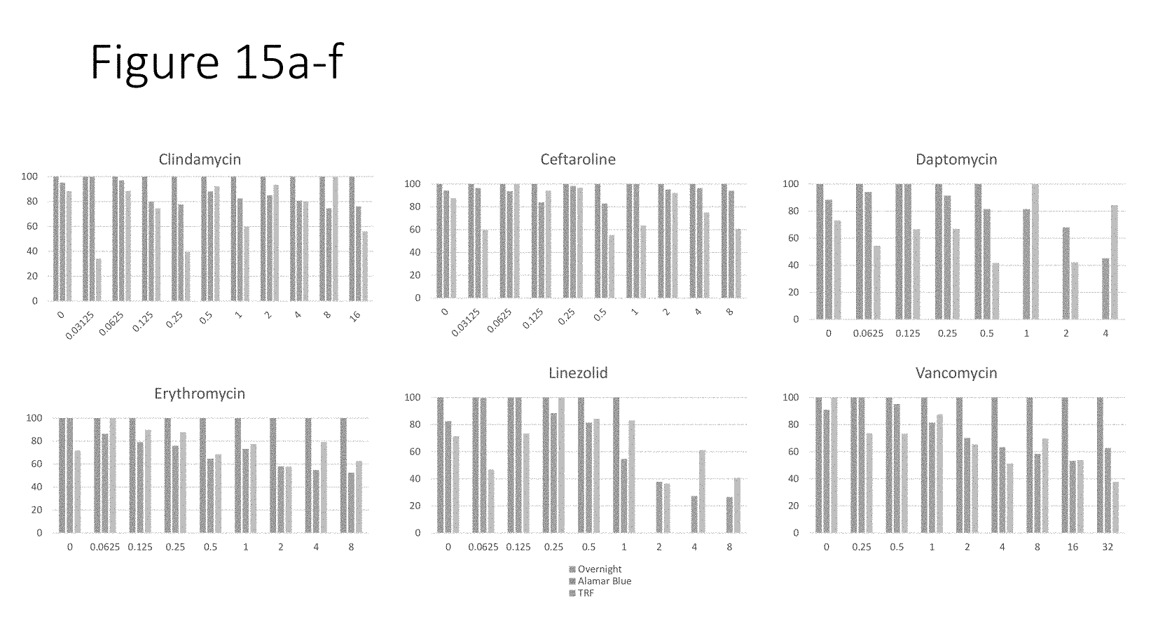

The positive blood culture (PBC) was removed from the blood culture machine. 6 mL of PBC was added to a 15 mL conical tube. Tubes were centrifuged at 250 RCF for 5 minutes. 5 mL supernatant was moved to a clean tube. The tubes were centrifuged at 1350 RCF for 5 minutes. Supernatant was aspirated, 5 mL saline was added, and tubes were vortexed. Tubes were centrifuged at 1350 RCF for 5 minutes. Supernatant was aspirated, pellets were resuspended with 0.5 mL saline. The resuspension was then treated as a 0.5 McFarland, and used to inoculate an AST assay. Representative data from this process can be seen in FIGS. 14-15

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments described herein. Such equivalents are intended to be encompassed by the following claims.

1-33. (canceled)

34. An automated method for preparing a sample suspected of comprising pathogenic microorganisms derived from a patient for antimicrobial susceptibility testing comprising the steps of:
a. applying a relative centrifugal force (RCF) of <1000×g to a sample, thereby producing a particulate depleted supernatant;
b. lysing at least one patient cell or platelet in the particulate depleted supernatant, thereby producing a patient cell depleted supernatant;
c. applying an RCF of >1000×g to the patient cell depleted supernatant, thereby forming a pellet;
d. collecting and resuspending the pellet;
e. optionally washing the pellet one or more times by repeating steps c and d; and
f. assessing a density of microbes in a saline or buffered solution.

35. The method of claim 34, wherein the lysing is done by a lytic reagent including, but not limited to, saponins, tritons, tweens, ammonium chloride, sorbitan esters, nonionic polyoxyethylene surfactants.

36. (canceled)

37. The method of claim 34, wherein the lytic reagent comprises, but is not limited to, sodium polyanethole sulfate (SPS), polypropylene glycol (PPG), potassium carbonate, potassium bicarbonate, N-cyclohexyl-3-aminopropanesulfonic acid (CAPS), ethylenediaminetetraacetic acid (EDTA), sodium chloride.

38. The method of claim 34, wherein the lytic reagent comprises saponin, SPS, and PPG.

39. (canceled)

40. The method of claim 34, wherein the lytic reagent comprises ammonium chloride and potassium bicarbonate and EDTA or EDTA sodium salt.

41. The method of claim 34, wherein the lytic reagent comprises Brij 97 and CAPS.

42. The method of claim 34, wherein the pellet is not washed.

43-46. (canceled)

47. The method of claim 34, wherein the cells in the resuspended pellet are assessed optically at one or more wavelengths between 500 nm and 700 nm.

48. The method of claim 34, wherein the cells are assessed by a spectrometer, a spectrophotometer, or a nephelometer.

49-51. (canceled)

52. The method of claim 34, wherein the first RCF applied is 500 RCF.

53. The method of claim 34, wherein the second RCF applied is 2370 RCF.

54. The method of claim 34, wherein the third RCF applied is 2370 RCF.

55. The method of claim 34, wherein the fourth RCF applied is 2370 RCF.

56. The method of claim 34, wherein one or more RCFs are applied at a fixed angle of 15°, 30°, 45°, 60°, 75°, <90°.

57. The method of claim 34, wherein the method is performed on positive blood cultures from continuous monitoring systems.

58. The method of claim 34, wherein the method is performed on cultures that turned positive ≤48 hours after introduction into the continuous monitoring blood culture system.

59. The method of claim 34, wherein the volume of the sample derived from the patient is less than 20 mL, 15 mL, 10 mL.

61-88. (canceled)

89. A method for preparing a sample suspected of comprising pathogenic microorganisms derived from a patient for antimicrobial susceptibility testing comprising the steps of:
a. centrifuging at <1000×g;
b. collecting the supernatant from step a, followed by centrifuging at >1000×g;
c. cleaning the pellet resulting from step b;
d. resuspending the pellet; and
e. inoculating microbial cells from the resuspended pellet into each of a plurality of wells of an AST cassette.

90. The method of claim 89, further comprising incubating the resuspended cells under conditions promoting microorganism growth.

91. (canceled)

92. (canceled)

93. The method of claim 89, wherein the pellet is resuspended in a growth media.

94. The method of claim 89, wherein the sample is aliquoted into two or more independent reservoirs after pellet resuspension.

95. The method of claim 89, wherein the measurement of cell concentration is performed optically by absorbance or nephelometry.

96. The method of claim 89, wherein the sample is urine, blood, synovial fluid, cerebrospinal fluid, sputum, bronchoalveolar lavage, nasal swab, or wound swab.

97-107. (canceled)

108. A method for preparing a sample suspected of comprising pathogenic microorganisms derived from a patient for antimicrobial susceptibility testing comprising the steps of:
a. applying a first relative centrifugal force to a sample, thereby forming a first pellet and a first supernatant;
b. applying a second relative centrifugal force to the first supernatant, thereby a second pellet and a second supernatant;
c. cleaning the second pellet resulting from step b;
d. resuspending the second pellet in a buffered solution; and
e. measuring a concentration of microorganisms resuspended in the buffered solution.

109. The method of claim 108, further comprising incubating the resuspended microorganisms under conditions promoting microorganism growth.

110. The method of claim 108, wherein the first relative centrifugal force is lower than the second relative centrifugal force.

111. (canceled)

112. (canceled)

113. The method of claim 108, wherein the pellet is resuspended in a growth media.

114. The method of claim 108, wherein the sample is aliquoted into two or more independent reservoirs after pellet resuspension.

115. The method of claim 108, wherein the measurement of cell concentration is performed optically by absorbance or nephelometry.

116. The method of claim 108, wherein the sample is urine, blood, synovial fluid, cerebrospinal fluid, sputum, bronchoalveolar lavage, nasal swab, or wound swab.

117-119. (canceled)

120. The method of claim 108, wherein growth determination follows claim 109.

121-123. (canceled)
